(19) United States
(12) Patent Application Publication
(10) Pub. No.: US 2022/0175678 A1
(43) Pub. Date: Jun. 9, 2022

(54) FORMULATIONS FOR ENCAPSULATION AND BIOAVAILABILITY IMPROVEMENT OF BIOACTIVE COMPOUNDS BASED ON NATURAL PLANT BASED MATERIALS

(71) Applicant: TECHNION RESEARCH & DEVELOPMENT FOUNDATION LIMITED, Haifa (IL)

(72) Inventor: Yoav David LIVNEY, Misgav (IL)

(21) Appl. No.: 17/602,866

(22) PCT Filed: Apr. 12, 2020
(86) PCT No.: PCT/IL2020/050435

§ 371 Date: Oct. 11, 2021

Related U.S. Application Data
(60) Provisional application No. 62/832,377, filed on Apr. 11, 2019.

(51) Int. Cl. A61K 9/16; A61K 31/122; A61K 36/81; A61K 36/9066; A61K 9/107; A61K 9/19; A23L 33/115

(52) U.S. Cl. CPC A61K 9/1664; A61K 31/122; A61K 36/81; A23L 33/115; A61K 9/107; A61K 9/19; A61K 36/9066

(57) ABSTRACT

Particles comprising a plant protein shell and encapsulating hydrophobic compounds and optionally also bioavailability enhancers, are provided. Further provided is a composition and a kit comprising the particle and methods of preparation thereof. Methods of use, such as for enhancing the bioavailability of the hydrophobic compound, are also provided.

### CROSS REFERENCE TO RELATED APPLICATIONS

This application claims the benefit of priority of U.S. Provisional Patent Application No. 62/832,377 filed Apr. 11, 2019 entitled “FORMULATION FOR SOLUBILIZATION OF BIOACTIVE COMPOUNDS”, the contents of which is incorporated herein by reference in their entirety.

### FIELD OF INVENTION

The present invention provides a formulation for improving solubilization and bioavailability of hydrophobic and lipophilic compounds.

### BACKGROUND OF THE INVENTION

Numerous bioactive compounds, including nutraceuticals and drugs, are poorly bioavailable, due to their poor solubility, and often also due to the action of either efflux transporters, like P-gp, or of detoxification enzymes, like Cytochrome P 450 enzyme family, or both.

Encapsulation is an expanding technology, with great potential in several areas, including the pharmaceutical and food industries. The delivery of bioactive compounds into the human body is highly affected by particle size, thus nanoencapsulation is an option to enhance bioavailability of such compounds. Nanoencapsulation of bioactive components, with poor solubility in aqueous solutions, can improve their dispersibility in water, and therefore also their bioavailability and bioactivity.

Protein-based nano-encapsulation technologies have been found effective in protecting the encapsulated bioactive during thermal treatment, exposure to UV or visible light, low pH, and during shelf life and digestion. Undesired sensory attributes like bitterness and astringency were masked, and the high bioavailability of nano-encapsulated nutraceuticals was clinically demonstrated in humans. Bioavailability of vitamin D was found to be comparably high in protein nanoparticles as in milk fat in a clinical study. However, there is still lack of natural, plant passed, non-allergenic encapsulation materials and techniques, which may not only improve dispersibility, and protect the bioactives, but also improve their bioavailability.

Astaxanthin (AX) (3,3′-dihydroxy-β-β-carotene-4,4′-dione) is a xanthophyll carotenoid found mainly in algae and marine animals, notably salmon, conferring their characteristic red-orange color. It can be synthesized only by few microorganisms, especially the green microalga Haematococcus Pluvialis.

To improve the bioavailability of AX several approaches were evaluated in order to find appropriate biomaterials, which would solubilize or disperse hydrophobic compounds such as AX in water, and facilitate its intestinal absorption.

The nutritional value of PP has been shown to be greater than that of other vegetable and cereal proteins, higher than casein and comparable to the nutritional value of whole egg. PP is produced from widely available and inexpensive raw materials and is a by-product of potato starch production. Furthermore, PP is considerably amphiphilic and water-soluble, so that it can be particularly useful for the encapsulation and solubilization of hydrophobic compounds with low water-solubility. PP comprises several fractions, mainly patatin, protease inhibitors, and higher molecular weight proteins. In particular, the patatin or patatin-rich fraction is well digestible.

Lecithin (LEC) is a mixture of several phospholipids containing mainly phosphatidylcholine, phosphatidylethanolamine and phosphatidylinositol. In the food industry, LEC is widely used as an emulsifier, viscosity regulator, anti-spattering and dispersing agent. LEC is absorbed in the human body as a lipid, assisted by bile salts. The presence of fat, fatty acids and lipids in the formulation increases the excretion of bile salts, thus it may enhance the bioavailability of AX and other lipophilic compounds.

AX is a strong antioxidant due to its many conjugated double bonds. It exhibits antioxidant activity stronger than the activities of vitamin E and β-carotene, which is attributed to its strong reactive-oxygen-species quenching activity. Curcumin is a natural polyphenol compound, found in the turmeric plant. Curcumin aids in the management of oxidative and inflammatory conditions, metabolic syndrome, arthritis, anxiety, and hyperlipidemia. Most of these benefits can be attributed to its antioxidant and anti-inflammatory effects. A major limitation of AX and curcumin is a low bioaccessibility and bioavailability, which stems from the fact that both compounds are almost insoluble in an aqueous solution.

In some embodiments, dissolving AX in olive oil, followed by its emulsification with PPs, or dissolving AX in ethanol with lecithin, and then mixing with PP solution, was hypothesized to provide additional improvement of the bioavailability while still maintaining the consumer-friendly “All-Natural Ingredients” labeling. In some embodiments, bioavailability of a hydrophobic compound, such as AX may be enhanced by using a bioavailability enhancer, such as quercetin (QUE). Without being limited to any particular mechanism or theory, it is postulated that a hydrophobic compound, such as AX is metabolized primarily by hepatic cytochrome P-450 1A1/2. QUE was found to inhibit cytochrome P-450 activity, and also P-glycoprotein, the efflux transporter which inhibits the absorption of numerous hydrophobic compounds. Additionally, bioavailability enhancers such as curcumin and rutin were found to inhibit P-glycoprotein and cytochrome P-450. Hence, without being limited to any particular mechanism or theory, it was hypothesized that encapsulation of bioavailability enhancers, such as QUE within a core-shell particle may enhance bioavailability and/or bioaccessibility of a hydrophobic compound, by reducing both efflux of the hydrophobic compound back into the intestinal lumen by P-gp, and its elimination by intestinal and/or hepatic cytochrome P-450.

### SUMMARY OF THE INVENTION

The invention relates to a particle comprising a plant protein shell and encapsulating hydrophobic compounds, composition and a kit comprising same. The invention further relates to methods of preparation thereof, and methods for using same, such as for enhancing the bioavailability of the hydrophobic compound.

In one aspect of the invention, there is provided a particle, comprising a hydrophobic core and an amphiphilic shell, wherein the hydrophobic core comprises a hydrophobic compound, and a plant oil, wherein the amphiphilic shell comprises an amphiphilic plant protein, wherein the w/w ratio of the hydrophobic compound to the plant oil ranges from 0.001:1 to 1:1, and wherein the w/w ratio of the amphiphilic plant protein to the plant oil ranges from 10:1 to 1:10 of the particle.

In another aspect, there is provided a particle comprising a plant oil encapsulated in an amphiphilic shell, wherein the amphiphilic shell comprises an amphiphilic plant protein and a hydrophobic compound, wherein the w/w ratio of the hydrophobic compound to the plant protein ranges from 0.01:1 to 1:1, and wherein the w/w ratio of the plant protein to the plant oil ranges from 0.1:1 to 10:1.

In one embodiment, the hydrophobic compound has a low solubility in the plant oil and is optionally selected from the group consisting of: a phenolic compound, a tannin, a stilbene, a curcuminoid, a coumarin, a lignan, a quinone, or any combination thereof.

In one embodiment, the shell is a single layer shell.

In one embodiment, the hydrophobic compound is selected from the group consisting of: a carotenoid, a flavonoid, a phytosterol, an antioxidant, a phytoestrogen, a polyphenol, a cannabinoid, a hydrophobic drug, a hydrophobic nutraceutical, and anthocyanin or any combination thereof.

In one embodiment, the carotenoid is selected from the group consisting of: astaxanthin (AX), astaxanthin oleoresin (AX oleoresin), beta-carotene, cantaxanthin, lutein, vitamin A (retinol), zeaxanthin, beta-zeacaroten, lycopene, apocarotenal, bixin, paprika oleoresin, capsanthin, and capsorubin or any combination thereof.

In one embodiment, the plant oil is selected from the group consisting of: an olive oil, a triglyceride oil, a terpenoid oil, a citrus oil, a sunflower oil, a peanut oil, a soy oil, a rapeseed oil, a soybean oil, a palm oil, a cocoa butter, a rice bran oil, and limonene or any combination thereof.

In one embodiment, the amphiphilic plant protein is selected from the group consisting of: a potato protein, a sweet potato protein, a soy protein, a rice protein, a wheat protein, a legume protein, a cereal protein, an algal protein, a hydrolyzed soy protein, a hydrolyzed rice protein, a hydrolyzed wheat protein, a hydrolyzed cereal protein, a hydrolyzed algal protein and a hydrolyzed legume protein or any combination thereof.

In one embodiment, the hydrophobic core comprises AX and olive oil.

In one embodiment, a w/w ratio of the AX to the olive oil is between 0.001:1 and 0.1:1; and wherein a w/w ratio of the PP to the olive oil is between 10:1 and 1:1.

In one embodiment, the hydrophobic compound comprises the curcuminoid, and the plant oil comprises the olive oil.

In one embodiment, a w/w ratio of the curcuminoid to the olive oil is between 0.1:1 and 1:1; and wherein a w/w ratio of the PP to the olive oil is between 3:1 and 1:3.

In one embodiment, a size of the particle is between 0.1 to 50 μm.

In another aspect, there is provided a particle comprising a plant oil encapsulated in an amphiphilic shell, wherein the amphiphilic shell comprises an amphiphilic plant protein and a hydrophobic compound, wherein the w/w ratio of the hydrophobic compound to the plant protein ranges from 0.01:1 to 1:1, and wherein the w/w ratio of the plant protein to the plant oil ranges from 0.1:1 to 10:1.

In one embodiment, the shell is a single layer shell.

In one embodiment, the hydrophobic compound is a natural phenol selected from the group consisting of: a phenolic acid, a tannin, a stilbene, a curcuminoid, a coumarin, a lignan, a quinone, or any combination thereof.

In one embodiment, the amphiphilic plant protein is selected from the group consisting of: a potato protein, a sweet potato protein, a soy protein, a rice protein, a wheat protein, a legume protein, a cereal protein, an algal protein, a hydrolyzed soy protein, a hydrolyzed rice protein, a hydrolyzed wheat protein, a hydrolyzed cereal protein, a hydrolyzed algal protein and a hydrolyzed legume protein or any combination thereof.

In one embodiment, the plant oil is selected from the group consisting of: an olive oil, a triglyceride oil, a terpenoid oil, a citrus oil, a sunflower oil, a peanut oil, a soy oil, a rapeseed oil, a soybean oil, a palm oil, a cocoa butter, a rice bran oil, and limonene or any combination thereof.

In another aspect, there is provided a particle comprising a hydrophobic core and an amphiphilic shell, wherein the hydrophobic core comprises a hydrophobic compound, wherein the amphiphilic shell comprises a first layer and a second layer, wherein the first layer comprises a surfactant and the second layer comprises an amphiphilic plant protein, wherein the w/w ratio of the amphiphilic plant protein to the surfactant ranges from 1:1 to 500:1, and wherein the w/w ratio of the hydrophobic compound to the surfactant ranges from 0.01:1 to 1:10 of the particle.

In one embodiment, the hydrophobic compound (i) is selected from the group consisting of: carotenoid, a flavonoid, a phytosterol, an antioxidant, a phytoestrogen, a polyphenol, and anthocyanin or any combination thereof; or (ii) has a low solubility in the plant oil and is optionally selected from the group consisting of: a phenolic acid, a tannin, a stilbene, a curcuminoid, a coumarin, a lignan, a quinone, or any combination thereof.

In one embodiment, the plant oil is selected from the group consisting of: an olive oil, a triglyceride oil, a terpenoid oil, a citrus oil, a sunflower oil, a peanut oil, a soy oil, a rapeseed oil, a soybean oil, a palm oil, a cocoa butter, a rice bran oil, and limonene or any combination thereof.

In one embodiment, the plant oil comprises the olive oil; the hydrophobic compound comprises curcumin, AX or both; and the surfactant comprises lecithin.

In one embodiment, the amphiphilic plant protein is selected from the group consisting of: a potato protein (PP), a sweet potato protein, a soy protein, a rice protein, a wheat protein, a legume protein, a cereal protein, an algal protein, a hydrolyzed soy protein, a hydrolyzed rice protein, a hydrolyzed wheat protein, a hydrolyzed cereal protein, a hydrolyzed algal protein and a hydrolyzed legume protein or any combination thereof.

In one embodiment, the PP comprises patatin, a protease inhibitor, a phosphorylase, or any combination thereof.

In one embodiment, the particle of the invention further comprising a cryoprotectant, an anti-oxidant, a preservative, an organic solvent, a bioavailability enhancer or any combination thereof.

In another aspect, there is provided a composition comprising the particle of the invention, and an aqueous solution, wherein the hydrophobic compound is at a concentration of 1 to 10000 ppm in the composition.

In another aspect, there is provided a composition comprising the particle of the invention, wherein the composition is a powderous composition, having less than 1 w/w % water.

In one embodiment, the composition further comprises a cryoprotectant, an anti-oxidant, a preservative, a solvent, a bioavailability enhancer or any combination thereof.

In another aspect, there is provided a method for solubilizing a hydrophobic compound in an aqueous formulation, comprising: (i) mixing the hydrophobic compound and an oil at a w/w ratio ranging from 0.01:1 to 1:1 at 30-70° C., to obtain an oil phase, (ii) providing an aqueous solution comprising an aqueous phosphate buffer and an amphiphilic plant protein, (iii) adding the aqueous solution to the oil phase to obtain a final solution, (iv) homogenizing the final solution, thereby solubilizing the hydrophobic compound in the aqueous formulation.

In another aspect, there is provided a method for solubilizing a hydrophobic compound in an aqueous formulation, comprising: (i) mixing the hydrophobic compound and a surfactant with ethanol, to obtain an ethanolic solution, (ii) providing an aqueous solution comprising an aqueous phosphate buffer and an amphiphilic plant protein, (iii) adding the ethanolic solution to the aqueous solution, thereby solubilizing the hydrophobic compound in the aqueous formulation.

In another aspect, there is provided a method for solubilizing a hydrophobic compound in an aqueous formulation, comprising: (i) mixing the hydrophobic compound and ethanol to obtain an ethanolic solution, (ii) providing an aqueous solution comprising an aqueous phosphate buffer and an amphiphilic plant protein, (iii) adding the ethanolic solution to the aqueous solution, to obtain a combined aqueous solution, (iii) mixing the combined aqueous solution with an amphiphilic plant oil to form a mixture, (iv) homogenizing the mixture, thereby solubilizing the hydrophobic compound in the aqueous formulation.

In one embodiment, the method further comprises freeze-drying the aqueous formulation to obtain a powder, thereby obtaining a powderous composition comprising the hydrophobic compound.

In one embodiment, the method further comprises mixing the powderous composition with an aqueous solution, thereby obtaining a reconstituted aqueous formulation comprising the hydrophobic compound.

In another aspect, there is provided a method of supplementing a subject with a hydrophobic compound, comprising the step of administering to the subject a composition of the invention, thereby supplementing the subject with the hydrophobic compound.

In one embodiment, the method is for enhancing bioavailability of the hydrophobic compound.

In one embodiment, the hydrophobic compound is administered at a dosage of 1-600 mg/kg body weight of the subject.

In one embodiment, the subject is selected from a human subject and an animal subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: A graph depicting the particle size distribution of a formulation containing Astaxanthin oleoresin (AX, depicted “AST”) and potato protein (PP) at two different molar ratios, before and after freeze drying and reconstitution.

FIG. 2: A graph depicting the particle size distribution of 0.5 mM AX solution vs. 0.5 mM PP solution, before and after freeze drying and reconstitution.

FIG. 3: A graph depicting the particle size distribution of a formulation containing AX, sunflower lecithin (LEC) and PP at different molar ratios, before and after freeze-drying and reconstitution.

FIG. 4: A graph depicting the particle size distribution of the formulation containing AX and LEC vs. 0.5 mM PP solution, before and after freeze drying and reconstitution.

FIG. 5: A graph depicting the particle size distribution of AX-olive oil-PP emulsion (formulation 3) containing 2 or 4% olive oil after 1 or 4 homogenization passes.

FIG. 6: A graph depicting the particle size distribution of AX-olive oil-PP emulsion (formulation 3) containing 4% olive oil after 4 homogenization cycles, before and after freeze drying and reconstitution.

FIGS. 7A-D: Light microscopy images of AX particles. FIG. 7A. Free AX in buffer solution. FIG. 7B. AX in formulation 1. FIG. 7C. AX in formulation 2.

FIG. 7D. AX in formulation 3.

FIG. 8: A bar graph depicting in-vitro bio-accessibility of AX either in a free form (AX oleoresin) or solubilized in formulation 1 (AX-PP nanoparticle dispersion), formulation 2 (AX-LEC-PP emulsion), or formulation 3 (AX-olive oil-PP emulsion at a ratio of 1:3:4).

FIGS. 9A-B: Graphs depicting the particle size distribution of formulations 3a-c (AX-olive oil-PP emulsion at a ratio of 1:3:4; AX-olive oil-PP emulsion at a ratio of 1:2:3; and AX-olive oil-PP emulsion at a ratio of 1:3:4, including tapioca maltodextrin, respectively) made on industrial equipment. FIG. 9A. Formulations after production. FIG. 9B. Formulations after freeze-drying and reconstitution.

FIG. 10: A bar graph depicting in-vitro bio-accessibility of formulations 3a-c, including formulation 3c,L (up scaled batch of AX-olive oil-PP emulsion at a ratio of 1:3:4) which was produced at a larger scale for the clinical trial.

FIG. 11: A graph depicting plasma AX concentration vs. time after capsules intake. The presented values are mean values (n=13).

FIGS. 12A-B: Bar graphs depicting plasma AX concentration after administration of formulation 3c vs. AX oleoresin (at same AX dose). FIG. 12A. AX area under the curve (AUC), *P<0.0007. FIG. 12B. Maximal AX concentration in plasma, **P<0.0012. The presented values are mean values (n=13).

FIGS. 13A-C: Schematic representation of particle structures in formulations 1 to 3. FIG. 13A. Schematic representation of a particle in formulation 3. FIG. 13B. Schematic representation of a particle in formulation 2. FIG. 13C. Schematic representation of a particle in the Curcumin-Olive oil-PP emulsion.

FIGS. 14A-C: Bar graphs and a table depicting the particle size distribution (nm) of the formulation containing curcumin-LEC-PP at various molar ratios. FIG. 14A: Particle size distribution within the formulation after production, before freeze-drying and reconstitution. FIG. 14B: Particle size distribution within the formulation after freeze-drying and reconstitution. FIG. 14C: A table summarizing the average particle diameter (nm) within the formulation before and after freeze-drying and reconstitution.

FIGS. 15A-C: Graphs and a table depicting the particle size distribution of a formulation containing curcumin-olive oil-PP emulsion at a weight ratio of 0.06:0.73:0.73, after a pre-homogenization, or after passing 1, 2, 3 and 4 homogenization cycles. FIG. 15A: Particle size distribution within the formulation after production, before freeze-drying and reconstitution. FIG. 15B: Particle size distribution within the formulation after freeze-drying and reconstitution. FIG. 15C: A table summarizing the average particle diameter (nm) within the formulation before and after freeze-drying and reconstitution.

FIG. 16: A bar graph depicting in-vitro bio-accessibility of the formulation comprising curcumin encapsulated by PP, versus non-encapsulated (free) curcumin.

FIG. 17: A bar graph depicting in-vitro protection of the encapsulated curcumin (PP-curcumin; curcumin-LEC-PP; and curcumin-olive oil (00)), versus non-encapsulated curcumin (CUR).

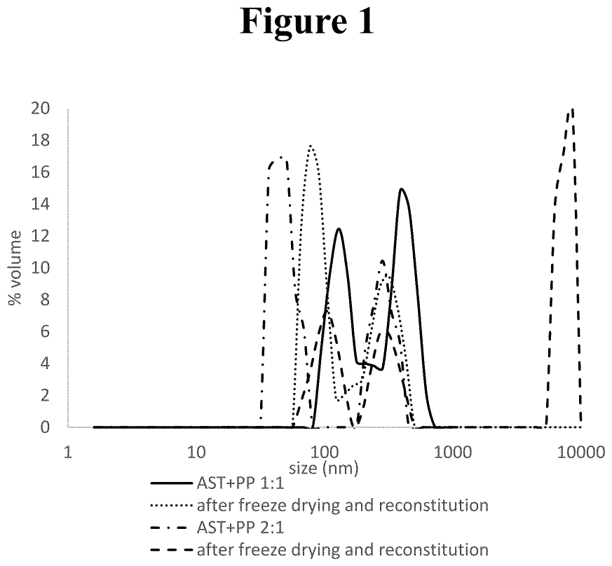

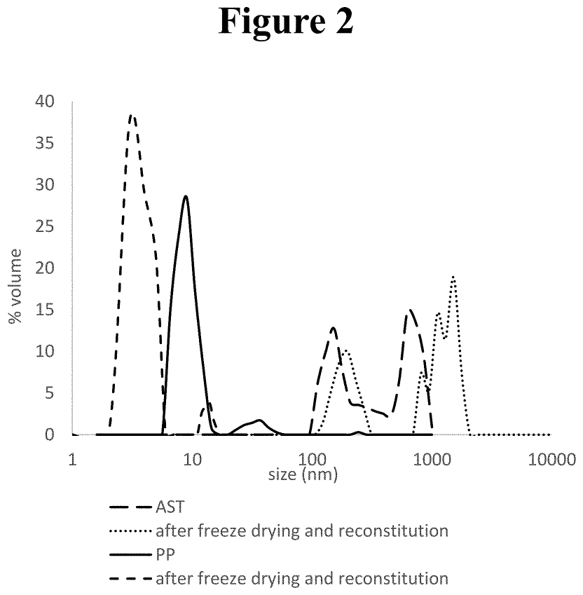

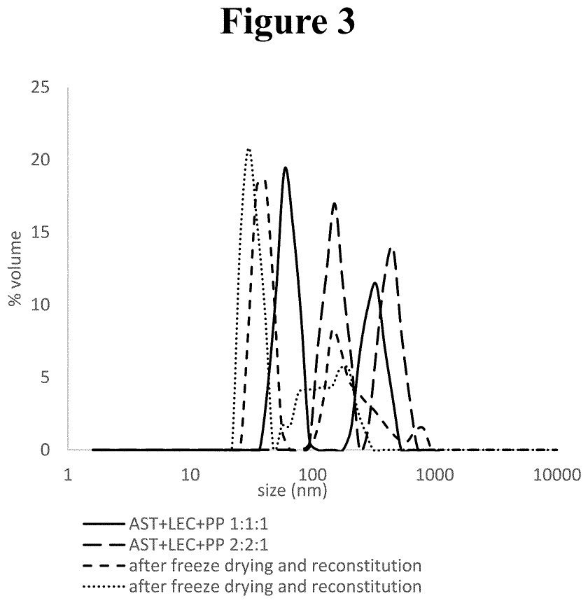

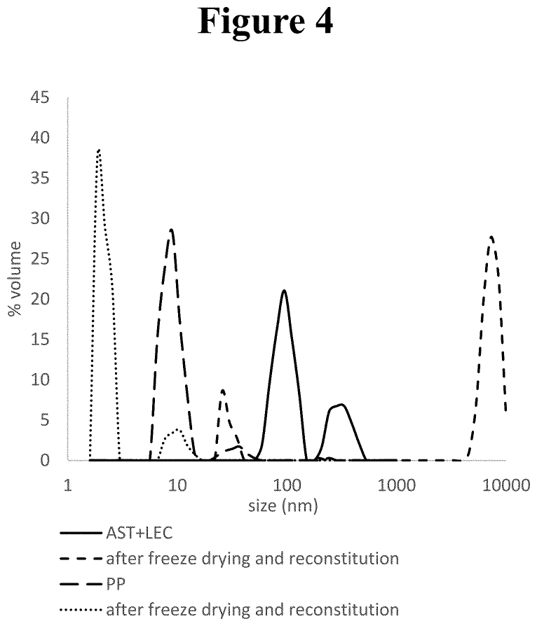

### DETAILED DESCRIPTION OF THE INVENTION

The invention relates to a particle comprising a plant protein shell and encapsulating hydrophobic compounds, composition and a kit comprising same. The invention further relates to methods of preparation thereof, and methods for using same, such as for enhancing the bioavailability of the hydrophobic compound.

The present invention is directed, in one embodiment, to a particle comprising an oil, a hydrophobic compound, and a plant protein. In another embodiment, provided herein a composition or a kit comprising a particle as described herein. In one embodiment, the particle is a water-dispersible particle. In one embodiment, the particle consists a shell and a core.

In another embodiment, provided herein is a particle, comprising a hydrophobic core and an amphiphilic shell, wherein the hydrophobic core comprises a hydrophobic compound, and a plant oil, wherein the amphiphilic shell comprises an amphiphilic plant protein, and wherein the weight/weight (w/w) ratio of the hydrophobic compound to the plant oil ranges from 0.001:1 to 1:1, and wherein the w/w ratio of the amphiphilic plant protein to the plant oil ranges from 10:1 to 1:10.

In another embodiment, provided herein is a particle comprising a hydrophobic core and an amphiphilic shell, wherein the hydrophobic core comprises a hydrophobic compound, wherein the amphiphilic shell comprises a first layer and a second layer, wherein the first layer comprises a surfactant and the second layer comprises an amphiphilic plant protein, wherein the w/w ratio of the amphiphilic plant protein to the surfactant ranges from 1:1 to 500:1, and wherein the w/w ratio of the hydrophobic compound to the surfactant ranges from 0.01:1 to 1:1. In one embodiment, the second layer is devoid of a surfactant. In one embodiment, a surfactant as described herein excludes plant protein. In one embodiment, the first layer is devoid of a plant protein.

In another embodiment, provided herein is a particle comprising a plant oil encapsulated in an amphiphilic shell, wherein the amphiphilic shell comprises an amphiphilic plant protein and a hydrophobic compound, wherein the w/w ratio of the hydrophobic compound to the plant protein ranges from 0.01:1 to 1:1, and wherein the w/w ratio of the plant protein to the plant oil ranges from 0.1:1 to 10:1 of the composition.

In one embodiment, provided herein is a method for encapsulating a hydrophobic compound within a particle, comprising solubilizing the hydrophobic compound in a plant oil and encapsulating the plant oil comprising the solubilized hydrophobic compound within an amphiphilic shell of the particle as described herein.

In another embodiment, provided herein, is a method for encapsulating a hydrophobic compound within a particle, comprising providing an ethanolic solution of a surfactant and the hydrophobic compound, and adding the ethanolic solution into an aqueous solution comprising a plant protein, thereby encapsulating the hydrophobic compound within an amphiphilic shell of the particle as described herein.

In another embodiment, provided herein, is a method for encapsulating a hydrophobic compound within a particle, comprising mixing an ethanolic solution of the hydrophobic compound with an aqueous solution comprising a plant protein, and adding a plant oil, thereby encapsulating the hydrophobic compound within an amphiphilic shell of the particle, as described herein.

In another embodiment, provided herein is a method for solubilizing a hydrophobic compound encapsulated within a particle in an aqueous solution, comprising contacting an aqueous solution with the particle as described herein.

In some embodiments, the present invention provides a method for enhancing the bioavailability of a hydrophobic compound in a subject, by administering a composition as described herein to the subject.

### Particle

In some embodiments, the particle as described herein is within a composition of the present invention. In some embodiments, the composition of the present invention comprises a solid particle. In some embodiments, the composition of the present invention comprises a stable particle. In some embodiments, the composition comprises an aqueous solution and a particle as described herein. In some embodiments, the composition comprises water and a particle as described herein.

In some embodiments, the hydrophobic core comprises a liquid oil. In some embodiments, the hydrophobic core comprises a plant oil. In some embodiments, the hydrophobic core further comprises a hydrophobic compound. In some embodiments, the hydrophobic compound is dissolved in the plant oil. In some embodiments, the hydrophobic compound is dispersed in the plant oil.

In some embodiments, the hydrophobic core comprises the plant oil and the hydrophobic compound. In some embodiments, the hydrophobic core comprises the plant oil and the hydrophobic compound in a liquid state.

In some embodiments, the hydrophobic core comprises the hydrophobic compound dissolved in an organic solvent. In some embodiments, the hydrophobic core comprises the hydrophobic compound dispersed in an organic solvent. In some embodiments the organic solvent is a non-polar solvent.

In some embodiments, the particle comprises less than 0.5% w/w ethanol. In some embodiments, the particle comprises less than 0.3% w/w ethanol. In some embodiments, the particle comprises less than 0.2% w/w ethanol. In some embodiments, the particle comprises less than 0.1% w/w ethanol.

In some embodiments, the amphiphilic shell is a single layer shell. In some embodiments, the amphiphilic shell is a double layer shell. In some embodiments, the amphiphilic shell comprises a first layer and a second layer.

In some embodiments, the single layer amphiphilic shell comprises an inner portion facing the hydrophobic core and an outer portion. In some embodiments, the inner portion of the shell in contact with the plant oil comprises a hydrophobic segment. In some embodiments, the hydrophilic segment is on the outer portion of the shell.

In some embodiments, the single layer shell comprises a plant protein. In some embodiments, the shell of the particle further comprises at least one additional plant protein. In some embodiments, the shell of the particle further comprises a mixture of plant proteins. In some embodiments, the shell further comprises a surfactant.

In some embodiments, the plant protein stabilizes the liquid core. In some embodiments, the plant protein is amphiphilic.

In some embodiments, a surfactant is a low molecular weight surfactant. In some embodiments, a surfactant is selected from the group consisting of: a monoglyceride, a diglyceride, a lecithin, a phospholipid, a fatty acid, a fatty acid salt, a bile salt, and a saponin or any combination thereof.

In some embodiments, a surfactant is a phospholipid. In some embodiments, the surfactant comprises a mixture of phospholipids.

Non-limiting examples of phospholipids include but are not limited to: sunflower lecithin, egg lecithin, egg phosphatidylglycerol, phosphatidic acid, lysolecithin, soy lecithin, hydrogenated soy lecithin, and sphingomyelin or any combination thereof.

In some embodiments, the surfactant is soluble in an organic solvent. In some embodiments, the surfactant is soluble in a polar solvent. In some embodiments, the surfactant is soluble in ethanol.

In some embodiments, the surfactant is sunflower lecithin. In some embodiments, the surfactant is an ethanol soluble fraction of sunflower lecithin (LEC).

In some embodiments, the shell is a double-layered shell comprising a first inner layer (i.e., surrounding the core) which comprises a surfactant, and a second outer layer comprising a plant protein.

In some embodiments, the hydrophobic segment of the first layer comprising a surfactant is in contact with the hydrophobic core of the particle. In some embodiments, the hydrophilic segment of the first layer is bound electrostatically to the second layer, comprising the plant protein. In some embodiments, the second layer stabilizes the first layer encapsulating the hydrophobic core. In some embodiments the hydrophilic segment of the second layer faces an aqueous solution, thus stabilizing the particle in the solution. In some embodiments, the second layer which faces an aqueous solution, is an outer layer of the shell.

In some embodiments, the plant protein encapsulates the liquid core comprising a hydrophobic compound as described herein. In some embodiments, the plant protein stabilizes the particle in an aqueous solution.

In some embodiments, the particle comprises an oil-insoluble hydrophobic compound. In some embodiments, the single layer shell comprises the oil-insoluble hydrophobic compound bound to the plant protein. In some embodiments, the oil-insoluble hydrophobic compound bound to the plant protein faces a hydrophobic core comprising the plant oil. As used herein, the term oil-insoluble hydrophobic compound refers to a compound having a low oil-solubility, wherein the oil solubility of the oil-insoluble hydrophobic compound is as described herein.

### Plant Protein

In some embodiments, the plant protein is amphiphilic. In some embodiments, the plant protein is a natural compound selected from the group consisting of: a vegetable protein, a soy protein, a rice protein, a cereal protein, a wheat protein, a legume protein, papain, a rapeseed protein, an alfalfa protein, a hydrolyzed soy protein, a hydrolyzed rice protein, a hydrolyzed wheat protein, and a hydrolyzed legume protein, a chickpea protein, a pea protein, a lentil protein, a bean protein, an algal protein, a hydrolyzed algal protein or any combination thereof. In some embodiments, the plant protein is a vegetable protein.

Non-limiting examples of vegetable proteins include but are not limited to: a potato protein, a sweet potato protein, a pea protein, a chickpea protein, a lupine protein, or any combination thereof.

In some embodiments, the vegetable protein is soluble in an aqueous solution. In some embodiments, the aqueous solution is a buffered solution. In some embodiments, the pH value of the buffered solution ranges from 6 to 8.

In some embodiments, the vegetable protein is a potato protein (PP). In some embodiments, the PP is from a source of PP isolate. In some embodiments, the PP is from a source of PP isolate having over 80% crude protein weight per dry weight. In some embodiments, the PP is from a source of PP isolate having over 95% crude protein weight per dry weight. In some embodiments, the PP is a PP isolate having over 97% crude protein weight per dry weight.

In some embodiments, the PP is patatin. In some embodiments, the PP is a fraction comprising protease inhibitors. In some embodiments, the PP is a phosphorylase. In some embodiments, the PP, fractions of PPs and methods for obtaining the same are described in U.S. Pat. No. 8,465,911.

### Plant Oil

In some embodiments, the plant oil is a natural triacylglyceride. In some embodiments, the plant oil is selected from medium-chain triacylglyceride (MCT) oil, and short-chain triacylglyceride (SCT) oil. In some embodiments, the plant oil is a terpenoid oil.

Non-limiting examples of plant oils include but are not limited to: an olive oil, a sunflower oil, a safflower oil, a corn oil, a canola oil, a wheat germ oil, a peanut oil, a soy oil, a coconut oil, a vegetable oil, an orange oil, a citrus oil, limonene, or any combination thereof. In some embodiments, the plant oil is a refined olive oil.

As used herein, the term “olive oil” comprises any oil derived from olives. As used herein, the term “refining” encompasses a process of removal undesired substances, such as free fatty acids, oleanolic acid, pigments, odors, and off-flavor components from an oil.

In some embodiments, the olive oil is an ester of glycerol and a fatty acid, wherein the fatty acid comprises a partially unsaturated C₄-C₂₂ hydrocarbon.

### Hydrophobic Compound

In some embodiments, the composition of the present invention comprises a hydrophobic compound. In some embodiments, the hydrophobic compound is an active ingredient of the composition. In some embodiments, the hydrophobic compound is a bioactive compound.

In some embodiments, the hydrophobic compound is a lipophilic compound. In some embodiments, the hydrophobic compound is oil soluble. In some embodiments, the hydrophobic compound is soluble in a plant oil. In some embodiments, the hydrophobic compound is soluble in a non-polar organic solvent. In some embodiments, the hydrophobic compound is non-soluble in an aqueous solution. In some embodiments, the hydrophobic compound has a low solubility in an aqueous solution. In some embodiments, the hydrophobic compound is oil insoluble. In some embodiments, the hydrophobic compound is substantially oil insoluble. In some embodiments, the hydrophobic compound is soluble in a polar organic solvent. In some embodiments, the hydrophobic compound is soluble in ethanol. In some embodiments, the organic solvent is a terpenoid oil.

In some embodiments, the hydrophobic compound has maximal aqueous solubility below 1 g/l. In some embodiments, the hydrophobic compound has maximal aqueous solubility below 0.5 g/l. In some embodiments, the hydrophobic compound has maximal aqueous solubility below 0.1 g/l. In some embodiments, the hydrophobic compound has maximal aqueous solubility below 0.01 g/l.

In some embodiments, the hydrophobic compound is an oil-soluble hydrophobic compound having a plant-oil solubility of at least 1 g/l, at least 0.7 g/l, at least 0.5 g/l, at least 0.3 g/l, at least 0.2 g/l, including any range or value therebetween.

In some embodiments, a solubility of the oil-soluble hydrophobic compound within a plant-oil is at most 100 g/l, at most 70 g/l, at most 50 g/l, at most 30 g/l, at most 20 g/l, at most 10 g/l, at most 5 g/l, at most 3 g/l, at most 1 g/l, at most 0.7 g/l, at most 0.5 g/l, at most 0.3 g/l, including any range or value therebetween.

In some embodiments, the oil-soluble hydrophobic compound is selected from the group consisting of: a carotenoid, a natural phenol (e.g. resveratrol), a vitamin, a hydrophobic vitamin (e.g. A, D, E, K), a cannabinoid, a hydrophobic drug, a polyunsaturated fatty acid (e.g. an omega-3 fatty acid), a phytosterol, a nutraceutical (e.g. co-Q10, genistein, daidzein, curcumin), an antioxidant, a phytoestrogen, a polyphenol, an anthocyanin, taurine or any combination thereof. Oil-soluble cannabinoids (including but not limited to THC and CBD and/or derivatives thereof) and hydrophobic drugs are well-known in the art.

In some embodiments, the hydrophobic compound is a carotenoid. Non-limiting examples of carotenoids include but are not limited to: astaxanthin (AX), astaxanthin oleoresin (AX oleoresin), beta-carotene, alpha-carotene, cantaxanthin, lutein, zeaxanthin, beta-zeacaroten, lycopene, apocarotenal, bixin, paprika oleoresin, capsanthin, vitamin A (retinol), cap sorubin or any combination thereof.

In some embodiments, the compound is an oil-insoluble compound. In some embodiments, the oil-insoluble compound or the hydrophobic compound is a natural phenol. Natural phenols (e.g. oil-insoluble phenols) are well-known in the art. Non-limiting examples of natural phenols include but are not limited to: phenolic acids, flavonoids, tannins, stilbenes, curcuminoids (e.g. curcumin), coumarins, lignans, quinones or any combination thereof. In some embodiments, the oil-insoluble compound comprises an oil-insoluble nutraceutical and/or an oil-insoluble drug.

In some embodiments, the oil insoluble compound has a plant-oil solubility of at least 0.001 g/l, at least 0.005 g/l, at least 0.01 g/l, at least 0.03 g/l, at least 0.05 g/l, at least 0.07 g/l, at least 0.1 g/l, at least 0.15 g/l, at least 0.17 g/l, at least 0.2 g/l, at least 0.25 g/l, at least 0.3 g/l, at least 0.5 g/l, including any range or value therebetween.

In some embodiments, a solubility of the oil-soluble hydrophobic compound within a plant-oil is at most 5 g/l, at most 3 g/l, at most 2 g/l, at most 1 g/l, at most 0.1 g/l, at most 0.5 g/l, at most 0.3 g/l, at most 0.2 g/l, at most 0.08 g/l, at most 0.05 g/l, at most 0.03 g/l, at most 0.01 g/l, including any range or value therebetween.

In some embodiments, the oil insoluble compound or the hydrophobic compound is a curcuminoid. Non-limiting examples of curcuminoids include but are not limited to curcumin, a derivative of curcumin (e.g. tetrahydrocurcumin, hexahydrocurcumin, curcumin sulfate, dihydrocurcumin, curcumin glucuronide or any combination thereof.

In some embodiments, the hydrophobic compound is AX oleoresin. In some embodiments, the w/w concentration of AX in the AX oleoresin is 10%.

### Composition

In some embodiments, the composition is a formulation. In some embodiments, the composition is a dispersion. In some embodiments, the particle is dispersed in an aqueous solution. In some embodiments, the hydrophilic segment of the particle shell forms bonding interaction with water molecules, thereby stabilizing the particle in the formulation.

In some embodiments, the hydrophobic segment of the particle shell is a dispersant, preventing from particles to agglomerate. In some embodiments, the formulation comprises the hydrophobic compound.

In some embodiments, the particle comprises a first layer and a second layer. In some embodiments, the second layer comprising the plant protein forms bonding interaction with water molecules, thereby stabilizing the particle in the formulation. In some embodiments, the formulation further comprises a polar solvent. In some embodiments, the polar solvent is a water miscible solvent. In some embodiments, the polar solvent is ethanol.

In some embodiments, the formulation comprises less than 0.5% w/w ethanol. In some embodiments, the formulation comprises less than 0.3% w/w ethanol. In some embodiments, the formulation comprises less than 0.2% w/w ethanol. In some embodiments, the formulation comprises less than 0.1% w/w ethanol. In some embodiments, the formulation comprises less than 5% w/w ethanol. In some embodiments, the formulation comprises less than 8% w/w ethanol. In some embodiments, the formulation comprises less than 10% w/w ethanol. In some embodiments, the formulation comprises less than 12% w/w ethanol. In some embodiments, the formulation comprises less than 15% w/w ethanol. In some embodiments, the formulation comprises less than 20% w/w ethanol.

In some embodiments, the formulation or the particle of the invention further comprises a component selected from the group consisting of: a cryoprotectant, an anti-oxidant, a preservative, a solvent, a bioavailability enhancer, or any combination thereof.

Non-limiting examples of bioavailability enhancers include but are not limited to: quercetin (QUE), rutin, hesperidin, curcumin, piperine, or any combination thereof.

In some embodiments, the particle of the invention further comprises a bioavailability enhancer at a w/w concentration between 0.1 and 10%, between 0.1 and 0.5%, between 0.5 and 1%, between 1 and 2%, between 2 and 5%, between 5 and 7%, between 7 and 10%, between 10 and 15%, between 15 and 20%, including any range or value therebetween.

Without being limited to any particular mechanism or theory, it was hypothesized that encapsulation of bioavailability enhancers, such as QUE within a core-shell particle may enhance bioavailability and/or bio-accessibility of the hydrophobic compound, wherein the hydrophobic compound as described herein.

In some embodiments, the bioavailability enhancer substantially enhances bioavailability and/or bio-accessibility of the hydrophobic compound, wherein substantially is as described herein. In some embodiments, the bioavailability enhancer enhances bioavailability and/or bio-accessibility of the hydrophobic compound by reducing (i) efflux of the hydrophobic compound back into the intestinal lumen by P-gp, (ii) by elimination of the hydrophobic compound by intestinal and/or hepatic cytochrome P-450 or by a combination of (i) and (ii).

In some embodiments, the formulation is an emulsion. In some embodiments, the formulation is an oil-in-water (o/w) emulsion.

In some embodiments, the o/w emulsion comprises the particle dispersed in the aqueous solution. In some embodiments, the o/w emulsion comprises the hydrophobic compound dissolved in the hydrophobic core of the particle. In some embodiments, the o/w emulsion comprises the hydrophobic compound dispersed in the hydrophobic core of the particle. In some embodiments, the hydrophobic compound is in the shell of the particle. In some embodiments, the emulsion comprises the hydrophobic compound bound to the shell of the particle.

In some embodiments, the o/w emulsion comprising the aqueous solution, the plant protein and the plant oil is used for solubilization of the hydrophobic compound. In some embodiments, the o/w emulsion comprising the surfactant, ethanol, the aqueous solution, and the plant protein is used for solubilization of the hydrophobic compound. In some embodiments, the o/w emulsion comprising the aqueous solution, ethanol, the plant protein and the plant oil is used for solubilization of the hydrophobic ethanol soluble compound. In some embodiments, the o/w emulsion enhances solubility of the hydrophobic compound. In some embodiments, the o/w emulsion enhances solubility of the hydrophobic compound in the aqueous solution.

In some embodiments, the o/w emulsion comprises water. In some embodiments, the o/w emulsion comprises a buffer as an aqueous solution. In some embodiments, the o/w emulsion comprises an aqueous buffered solution. In some embodiments, the o/w emulsion comprises a phosphate buffer. In some embodiments, the pH value of a phosphate buffer ranges from 2 to 8. In some embodiments, the pH value of a phosphate buffer ranges from 6.5 to 7. In some embodiments, the pH value of a phosphate buffer ranges from 6.8 to 7.5. In some embodiments, the pH value of a phosphate buffer ranges from 2 to 3. In some embodiments, the pH value of a phosphate buffer ranges from 3 to 4. In some embodiments, the pH value of a phosphate buffer ranges from 4 to 6.

In some embodiments, the o/w emulsion comprises at least 70% w/w water. In some embodiments, the o/w emulsion comprises at least 75% w/w water. In some embodiments, the o/w emulsion comprises at least 85% w/w water. In some embodiments, the o/w emulsion comprises at least 90% w/w water. In some embodiments, the o/w emulsion comprises at least 95% w/w water. In some embodiments, the o/w emulsion comprises at least 98% w/w water.

### Micro Particles Containing Oil-Soluble Hydrophobic Compound

In another aspect, there is a particle comprising a hydrophobic core and an amphiphilic shell, wherein the hydrophobic core comprises a hydrophobic compound and a plant oil, and wherein the amphiphilic shell comprises an amphiphilic plant protein. In some embodiments, the hydrophobic compound is an oil soluble compound. In some embodiments, the particle comprises an oil soluble hydrophobic compound, the plant oil, and the plant protein. In some embodiments, the oil soluble hydrophobic compound is as described hereinabove. In some embodiments, the hydrophobic core of the particle comprises a carotenoid (e.g. AX) and a plant oil (e.g. olive oil). In some embodiments, the hydrophobic core of the particle comprises a carotenoid (e.g. AX) and a plant oil (e.g. olive oil); and the amphiphilic shell of the particle comprises PP.

In some embodiments, the hydrophobic core is encapsulated by the shell comprising the plant protein. In some embodiments, the hydrophobic core comprising the hydrophobic compound encapsulated by the shell comprising the plant protein. In some embodiments, the hydrophobic compound is homogenously distributed (dissolved or dispersed) within the hydrophobic core.

In some embodiments, the shell is devoid of an additional amphiphilic compound. In some embodiments, the shell is devoid of an additional protein. In some embodiments, the shell is devoid of an additional plant protein. In some embodiments, the shell is devoid of a surfactant. In some embodiments, the shell is a single layer shell.

In some embodiments, the shell is substantially devoid of the hydrophobic compound, wherein substantially is at least 70%, at least 80%, at least 85%, at least 90%, at least 91%, at least 92%, at least 93%, at least 95%, at least 97%, at least 98%, at least 99% w/w of the shell.

In some embodiments, at least 70%, at least 80%, at least 85%, at least 90%, at least 91%, at least 92%, at least 93%, at least 95%, at least 97%, at least 98%, at least 99% w/w of the shell is composed of the amphiphilic plant protein. In some embodiments, at least 70%, at least 80%, at least 85%, at least 90%, at least 91%, at least 92%, at least 93%, at least 95%, at least 97%, at least 98%, at least 99% w/w of the hydrophobic core is composed of the hydrophobic compound (such as the oil-soluble hydrophobic compound).

In some embodiments, at least 70%, at least 80%, at least 85%, at least 90%, at least 91%, at least 92%, at least 93%, at least 95%, at least 97%, at least 98%, at least 99% w/w of the hydrophobic core is composed of a mixture (e.g. a solution or a dispersion) comprising hydrophobic compound and the plant oil.

In some embodiments, the w/w ratio of the hydrophobic compound to the plant oil in the particle ranges from 0.001:1 to 1:1. In some embodiments, the w/w ratio of the hydrophobic compound to the plant oil in the particle ranges from 0.015:1 to 0.1:1. In some embodiments, the w/w ratio of the hydrophobic compound to the plant oil in the particle ranges from 0.1:1 to 0.5:1. In some embodiments, the w/w ratio of the hydrophobic compound to the plant oil in the particle ranges from 0.5:1 to 1:1. In some embodiments, the w/w ratio of the hydrophobic compound to the plant oil in the particle ranges from 0.05:1 to 0.04:1. In some embodiments, the w/w ratio of the hydrophobic compound to the plant oil in the particle ranges from 0.1:1 to 0.05:1. In some embodiments, the w/w ratio of the hydrophobic compound to the plant oil in the particle ranges from 0.04:1 to 0.03:1. In some embodiments, the w/w ratio of the hydrophobic compound to the plant oil in the particle ranges from 0.03:1 to 0.02:1. In some embodiments, the w/w ratio of the hydrophobic compound to the plant oil in the particle ranges from 0.02:1 to 0.01:1. In some embodiments, the w/w ratio of the hydrophobic compound to the plant oil in the particle ranges from 0.01:1 to 0.005:1. In some embodiments, the w/w ratio of the hydrophobic compound to the plant oil in the particle ranges from 0.002:1 to 0.003:1.

In some embodiments, the w/w ratio of the hydrophobic compound to the plant oil in the particle is between 0.001:1 and 1:1, between 0.005:1 and 1:1, between 0.01:1 and 1:1, between 0.02:1 and 1:1, between 0.03:1 and 1:1, between 0.04:1 and 1:1, between 0.05:1 and 1:1, between 0.06:1 and 1:1, between 0.07:1 and 1:1, between 0.08:1 and 1:1, between 0.1:1 and 1:1, between 0.2:1 and 1:1, between 0.3:1 and 1:1, between 0.4:1 and 1:1, between 0.5:1 and 1:1, between 0.6:1 and 1:1, between 0.7:1 and 1:1, between 0.8:1 and 1:1, between 0.9:1 and 1:1, including any range or value therebetween.

In some embodiments the w/w ratio of the plant protein to the plant oil in the particle ranges from 10:1 to 1:10. In some embodiments the w/w ratio of the plant protein to the plant oil in the particle ranges from 1:1 to 4:1. In some embodiments the w/w ratio of the plant protein to the plant oil in the particle ranges from 3:2 to 4:1.5. In some embodiments, the w/w ratio of the plant protein to the plant oil in the particle ranges from 10:1 to 1:1. In some embodiments, the w/w ratio of the plant protein to the plant oil in the particle ranges from 1:1 to 2:1. In some embodiments, the w/w ratio of the plant protein to the plant oil in the particle ranges from 2:1 to 5:1. In some embodiments, the w/w ratio of the plant protein to the plant oil in the particle ranges from 10:1 to 5:1. In some embodiments, the w/w ratio of the plant protein to the plant oil in the particle ranges from 5:1 to 1:1. In some embodiments, the w/w ratio of the plant protein to the plant oil in the particle ranges from 1:1 to 5:1. In some embodiments, the w/w ratio of the plant protein to the plant oil in the particle ranges from 5:1 to 10:1.

In some embodiments, the w/w ratio of the plant protein to the plant oil within the particle is between 10:1 and 1:1, between 9:1 and 1:1, between 8:1 and 1:1, between 7:1 and 1:1, between 6:1 and 1:1, between 5:1 and 1:1, between 4:1 and 1:1, between 3:1 and 1:1, between 2:1 and 1:1, including any range or value therebetween.

In some embodiments, the w/w concentration of the plant protein in the emulsion ranges from 1 to 10%, from 2 to 6%, from 3 to 4%.

In some embodiments, the w/w concentration of the hydrophobic compound (e.g. oil-soluble hydrophobic compound, such as a carotenoid) within the particle is between 0.01 and 10%, between 0.01 and 0.05%, between 0.05 and 0.1%, between 0.1 and 0.3%, between 0.3 and 0.5%, between 0.5 and 1%, between 1 and 2%, between 2 and 3%, between 3 and 5%, between 5 and 7%, between 7 and 10%, between 10 and 20%, including any range or value therebetween.

In some embodiments, the total oil w/w concentration comprises the w/w concentration of the plant oil and the w/w concentration of the hydrophobic compound in the emulsion. In some embodiments, the total oil w/w concentration in the emulsion ranges from 1 to 10%, from 1 to 6%, from 1.5 to 2.5%, from 3 to 4.5%.

In some embodiments, the emulsion comprises the hydrophobic compound at the concentration of 1 to 10000 ppm. In some embodiments, the emulsion comprises the hydrophobic compound at the concentration of 1 to 5000 ppm. In some embodiments, the emulsion comprises the hydrophobic compound at the concentration of 1 to 3000 ppm. In some embodiments, the emulsion comprises the hydrophobic compound at the concentration of 1 to 2000 ppm. In some embodiments, the emulsion comprises the hydrophobic compound at the concentration of 1 to 1000 ppm. In some embodiments, the emulsion comprises the hydrophobic compound at the concentration of 100 to 1000 ppm. In some embodiments, the emulsion comprises the hydrophobic compound at the concentration of 300 to 1000 ppm. In some embodiments, the emulsion comprises the hydrophobic compound at the concentration of 300 to 2000 ppm. In some embodiments, the emulsion comprises the hydrophobic compound at the concentration of 500 to 3000 ppm.

In some embodiments, the particle size of the emulsion ranges from 0.1 to 50 μm. In some embodiments, the particle size of the emulsion ranges from 0.1 to 2 μm. In some embodiments, the particle size of the emulsion ranges from 0.2 to 3 μm. In some embodiments, the particle size of the emulsion ranges from 1 to 5 μm. In some embodiments, the particle size of the emulsion ranges from 5 to 8 μm. In some embodiments, the particle size of the emulsion ranges from 7 to 10 μm.

In some embodiments, the particle size is between 0.2 and 50 μm, between 0.2 and 1 μm, between 1 and 3 μm, between 3 and 5 μm, between 1 and 5 μm, between 5 and 10 μm, between 10 and 15 μm, between 15 and 20 μm, between 20 and 30 μm, between 30 and 50 μm including any range or value therebetween.

In some embodiments, the particle size as used herein, refers to a mean value. In some embodiments, the particle size as used herein, refers to a hydrodynamic diameter of the particle.

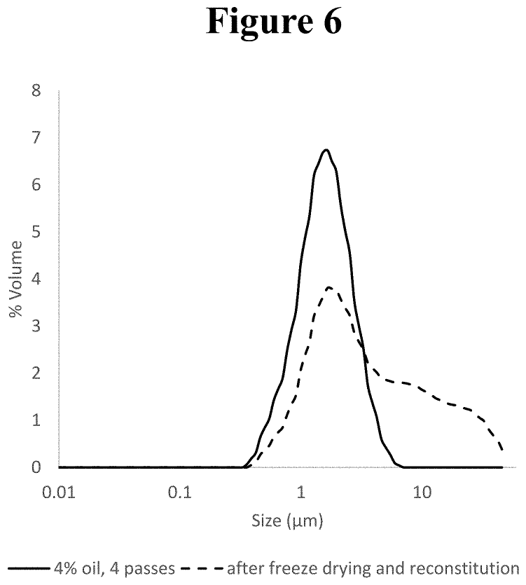

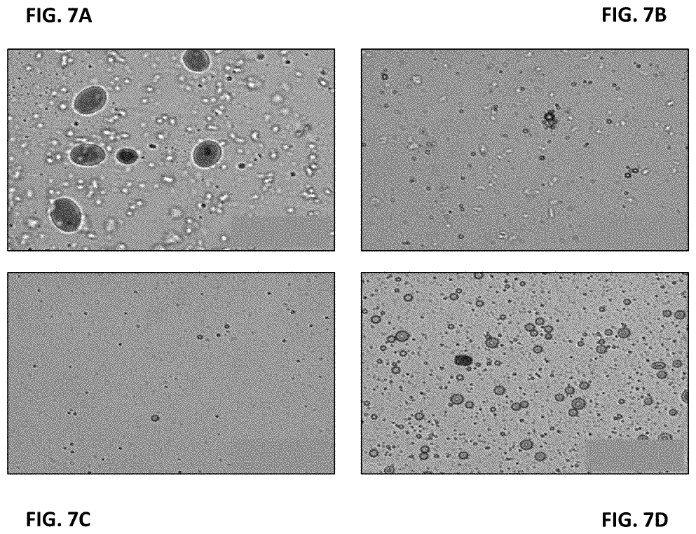

In some embodiments, the particle size upon reconstitution remains substantially the same as compared to the particle size before drying (as shown by FIG. 6), wherein substantially is as described herein.

In some embodiments, the emulsion comprises a cryoprotectant selected from the group consisting of: trehalose, a starch, a modified starch, and maltodextrin. In some embodiments, the cryoprotectant is tapioca maltodextrin.

In some embodiments, the w/w ratio of the cryoprotectant to the plant protein in the emulsion ranges from 1:2 to 1:200. In some embodiments, the w/w ratio of the cryoprotectant to the plant protein in the emulsion ranges from 1:2 to 1:200. In some embodiments, the w/w ratio of the cryoprotectant to the plant protein in the emulsion ranges from 1:2 to 1:10. In some embodiments, the w/w ratio of the cryoprotectant to the plant protein in the emulsion ranges from 1:10 to 1:20. In some embodiments, the w/w ratio of the cryoprotectant to the plant protein in the emulsion ranges from 1:20 to 1:50. In some embodiments, the w/w ratio of the cryoprotectant to the plant protein in the emulsion ranges from 1:50 to 1:100. In some embodiments, the w/w ratio of the cryoprotectant to the plant protein in the emulsion ranges from 1:100 to 1:200.

### Nanoparticles Containing Oil-Soluble Hydrophobic Compound

In another aspect, there is a particle comprising a hydrophobic core and an amphiphilic shell, wherein the hydrophobic core comprises a hydrophobic compound, wherein the amphiphilic shell comprises a first layer and a second layer, wherein the first layer comprises a surfactant and the second layer comprises an amphiphilic plant protein. In some embodiments, the w/w ratio of the amphiphilic plant protein to the surfactant within the particle ranges from 1:1 to 500:1, and wherein the w/w ratio of the hydrophobic compound to the surfactant within the particle ranges from 0.01:1 to 1:10. In some embodiments, the particle is a nano-particle. In some embodiments, the amphiphilic plant protein of the particle comprises potato protein, and the surfactant of the particle comprises lecithin.

In some embodiments, the hydrophobic core of the particle comprises an oil-soluble hydrophobic compound, an oil-insoluble hydrophobic compound or both. In some embodiments, the hydrophobic core comprises any of a carotenoid, a curcuminoid or both. In some embodiments, the amphiphilic shell of the particle comprises, the surfactant, and the plant protein, wherein the surfactant and the plant protein are as described herein.

In some embodiments, the amphiphilic shell of the particle comprises a plurality of layers. In some embodiments, the amphiphilic shell of the particle comprises two layers. In some embodiments, the amphiphilic shell comprises a first inner layer facing or in contact with the hydrophobic core. In some embodiments, the amphiphilic shell comprises a second outer layer in contact with the first layer. In some embodiments, the outer portion of the second layer faces the ambient and the inner portion of the second layer faces or is in contact with the first inner layer. In some embodiments, the first inner layer comprises the surfactant of the invention. In some embodiments, the first inner layer comprises lecithin. In some embodiments, the second outer layer comprises PP.

In some embodiments, the amphiphilic shell comprises the surfactant (e.g. lecithin) and the plant protein (e.g. PP) encapsulating the hydrophobic compound, wherein the hydrophobic compound is as described herein. In some embodiments, the hydrophobic chain of the surfactant is in contact with the hydrophobic compound or in contact with the hydrophobic core. In some embodiments, the hydrophilic head (a charged or a polar group such as a phosphate, carboxy or choline) of the surfactant is in contact with the second outer layer comprising the plant protein (e.g. PP). In some embodiments, the hydrophobic chain penetrates into the hydrophobic core.

In some embodiments, the surfactant and the plant protein form a single layer amphiphilic shell. In some embodiments, the surfactant and the plant protein form an interpenetrating network. In some embodiments, surfactant molecules are positioned in between the plant protein molecules. In some embodiments, surfactant molecules fill an empty space between the plant protein molecules, so as to form a uniform amphiphilic shell.

In some embodiments, at least a part of the surfactant and the plant protein are homogenously distributed within the amphiphilic shell. In some embodiments, the surfactant and the plant protein form a substantially homogenous amphiphilic shell, wherein substantially is as described herein.

In some embodiments, at least 1%, at least 5%, at least 10%, at least 15%, at least 20%, at least 25%, at least 30%, at least 40%, at least 50% w/w of the surfactant is mixed with or bound to the outer surface of the hydrophobic core.

In some embodiments, at most 1%, at most 5%, at most 10%, at most 15%, at most 20%, at most 25%, at most 30%, at most 40%, at most 50% w/w of the surfactant is mixed with or bound to the outer surface of the hydrophobic core.

In some embodiments, at least 10%, at least 15%, at least 20%, at least 25%, at least 30%, at least 40%, at least 50%, at least 60%, at least 70%, at least 80%, at least 90%, at least 95%, at least 97%, at least 99%, w/w of the surfactant is mixed with or bound to the second outer layer.

In some embodiments, at least 10%, at least 15%, at least 20%, at least 25%, at least 30%, at least 40%, at least 50%, at least 60%, at least 70%, at least 80%, at least 90%, at least 95%, at least 97%, at least 99%, w/w of the surfactant is mixed with or bound to the plant protein.

In some embodiments, at most 10%, at most 15%, at most 20%, at most 25%, at most 30%, at most 40%, at most 50%, at most 60%, at most 70%, at most 80%, at most 90%, at most 95%, at most 97%, at most 99%, w/w of the surfactant is mixed with or bound to the plant protein.

In some embodiments, the hydrophobic core substantially comprises the hydrophobic compound of the invention, wherein substantially is as described herein. In some embodiments, the amphiphilic shell substantially comprises the surfactant and the plant protein, wherein substantially is as described herein.

In some embodiments, the w/w ratio of the plant protein to the surfactant ranges from 1:1 to 500:1. In some embodiments, the w/w ratio of the plant protein to the surfactant ranges from 1:1 to 10:1. In some embodiments, the w/w ratio of the plant protein to the surfactant ranges from 10:1 to 15:1. In some embodiments, the w/w ratio of the plant protein to the surfactant ranges from 15:1 to 25:1. In some embodiments, the w/w ratio of the plant protein to the surfactant ranges from 25:1 to 50:1. In some embodiments, the w/w ratio of the plant protein to the surfactant ranges from 50:1 to 60:1. In some embodiments, the w/w ratio of the plant protein to the surfactant ranges from 60:1 to 100:1. In some embodiments, the w/w ratio of the plant protein to the surfactant ranges from 100:1 to 500:1.

In some embodiments, the particle encapsulating the oil-soluble hydrophobic compound is characterized by a w/w ratio of the plant protein to the surfactant being between 1:1 and 500:1, between 1:1 and 10:1, between 10:1 and 20:1, between 20:1 and 30:1, between 30:1 and 40:1, between 40:1 and 50:1, between 50:1 and 60:1, between 60:1 and 70:1, between 70:1 and 100:1, between 100:1 and 200:1, between 200:1 and 300:1, between 300:1 and 400:1, between 400:1 and 500:1, including any range or value therebetween. In some embodiments, the particle encapsulating the oil-soluble hydrophobic compound is characterized by a w/w ratio of the plant protein to the surfactant being between 10:1 and 60:1, wherein the oil-soluble compound is as described herein.

In some embodiments, the particle encapsulating the oil-insoluble hydrophobic compound is characterized by a w/w ratio of the plant protein to the surfactant being between 1:1 and 500:1, between 1:1 and 2:1, between 2:1 and 3:1, between 3:1 and 4:1, between 4:1 and 5:1, between 5:1 and 6:1, between 6:1 and 7:1, between 7:1 and 8:1, between 8:1 and 10:1, between 10:1 and 20:1, between 20:1 and 30:1, between 30:1 and 40:1, between 40:1 and 50:1, between 50:1 and 60:1, between 60:1 and 70:1, between 70:1 and 100:1, between 100:1 and 200:1, between 200:1 and 300:1, between 300:1 and 400:1, between 400:1 and 500:1, including any range or value therebetween. In some embodiments, the particle encapsulating the oil-insoluble hydrophobic compound is characterized by a w/w ratio of the plant protein to the surfactant being between 2:1 and 10:1, wherein the oil-insoluble compound is as described herein.

In some embodiments, the particle encapsulates at most 20%, at most 15%, at most 10%, at most 8%, at most 6%, at most 5%, at most 4%, at most 3%, at most 1%, at most 0.5%, at most 0.1% by weight of the oil-insoluble compound.

In some embodiments, the particle encapsulates at most 10%, at most 8%, at most 6%, at most 5%, at most 4%, at most 3%, at most 1%, at most 0.5%, at most 0.1% by weight of the oil-soluble compound.

In some embodiments, the w/w ratio of the hydrophobic compound to the surfactant ranges from 0.01:1 to 1:1. In some embodiments, the w/w ratio of the hydrophobic compound to the surfactant ranges from 0.01:1 to 0.05:1. In some embodiments, the w/w ratio of the hydrophobic compound to the surfactant ranges from 0.05:1 to 0.1:1. In some embodiments, the w/w ratio of the hydrophobic compound to the surfactant ranges from 0.1:1 to 0.5:1. In some embodiments, the w/w ratio of the hydrophobic compound to the surfactant ranges from 0.5:1 to 1:1.

In some embodiments, the w/w ratio of the hydrophobic oil-soluble compound (e.g. carotenoid) to the surfactant is between 0.01:1 and 1:1, between 0.01:1 and 0.05:1, between 0.05:1 and 0.1:1, between 0.1:1 and 0.3:1, between 0.3:1 and 0.5:1, between 0.5:1 and 0.7:1, between 0.7:1 and 0.8:1, between 0.8:1 and 1:1, including any range or value therebetween. In some embodiments, the w/w ratio of the hydrophobic oil-soluble compound (e.g. carotenoid) to the surfactant is between 0.5:1 and 1:1. In some embodiments, the molar ratio of the hydrophobic oil-soluble compound (e.g. carotenoid) to the surfactant is between 0.5:1 and 1.5:1

In some embodiments, the w/w ratio of the hydrophobic oil-insoluble compound (e.g. curcuminoid) to the surfactant is between 0.01:1 and 1:1, between 0.01:1 and 0.05:1, between 0.05:1 and 0.1:1, between 0.1:1 and 0.3:1, between 0.3:1 and 0.5:1, between 0.5:1 and 0.7:1, between 0.7:1 and 0.8:1, between 0.8:1 and 1:1, including any range or value therebetween. In some embodiments, the w/w ratio of the hydrophobic oil-insoluble compound (e.g. curcuminoid) to the surfactant is between 0.5:1 and 1:1. In some embodiments, the molar ratio of the hydrophobic oil-insoluble compound (e.g. curcuminoid) to the surfactant is between 0.5:1 and 1.5:1.

In some embodiments, the size of the nano-particle in the formulation is ranging from 10 to 1000 nm. In some embodiments, the size of the nano-particle in the formulation is ranging from 50 to 100 nm. In some embodiments, the size of the nano-particle in the formulation is ranging from 100 to 200 nm. In some embodiments, the size of the nano-particle in the formulation is ranging from 200 to 500 nm. In some embodiments, the size of the nano-particle in the formulation is ranging from 500 to 1000 nm.

In some embodiments, a size (or a mean hydrodynamic diameter) of the particle encapsulating the hydrophobic oil-insoluble compound (e.g. curcuminoid) is between 10 and 1000 nm, between 10 and 20 nm, between 20 and 30 nm, between 30 and 50 nm, between 50 and 60 nm, between 60 and 70 nm, between 70 and 100 nm, between 100 and 200 nm, between 100 and 150 nm, between 150 and 200 nm, between 200 and 300 nm, between 300 and 500 nm, between 500 and 1000 nm, including any range or value therebetween. In some embodiments, a size (or a mean hydrodynamic diameter) of the particle encapsulating the hydrophobic oil-insoluble compound (e.g. curcuminoid) is between 15 and 100 nm. In some embodiments, a diameter of the particle encapsulating the hydrophobic oil-insoluble compound is between 10 and 500 nm.

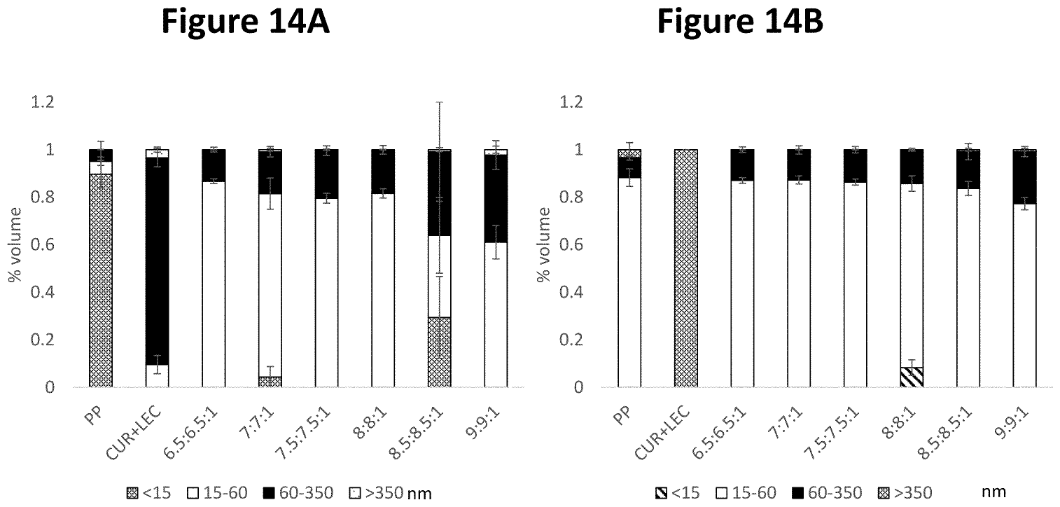

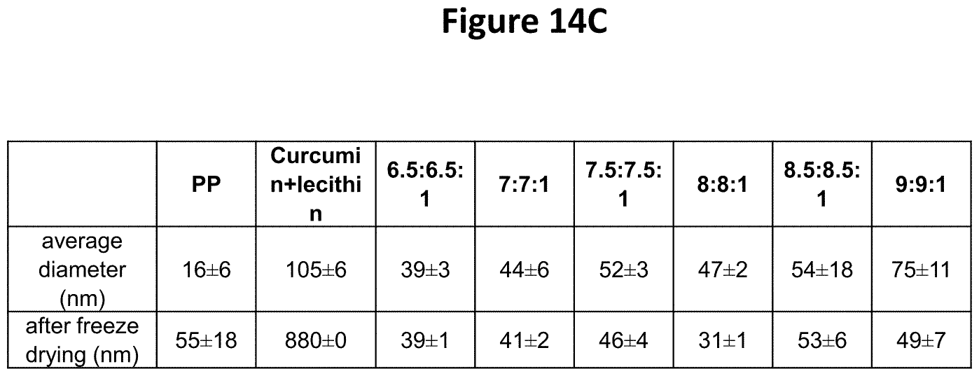

In some embodiments, a diameter of at least 50%, at least 60%, at least 70%, at least 75%, at least 80%, at least 83%, at least 85% of the particles encapsulating the hydrophobic oil-insoluble compound is between 15 and 60 nm (as exemplified by FIG. 14).

In some embodiments, a diameter of at most 50%, at most 40%, at most 30%, at most 25%, at most 20%, at most 15%, at most 10% of the particles encapsulating the hydrophobic oil-insoluble compound is between 60 and 350 nm (as exemplified by FIG. 14).

In some embodiments, a size (or a mean hydrodynamic diameter) of the particle encapsulating the hydrophobic oil-soluble compound (e.g. carotenoid) is between 10 and 1000 nm, between 10 and 20 nm, between 20 and 30 nm, between 30 and 50 nm, between 50 and 60 nm, between 60 and 70 nm, between 70 and 100 nm, between 100 and 200 nm, between 100 and 150 nm, between 150 and 200 nm, between 200 and 300 nm, between 300 and 400 nm, between 400 and 500 nm, between 500 and 700 nm, between 700 and 900 nm, between 900 and 1000 nm including any range or value therebetween. In some embodiments, a size (or a mean hydrodynamic diameter) of the particle encapsulating the hydrophobic oil-soluble compound (e.g. carotenoid) is between 30 and 500 nm.

In some embodiments, the size of the particle of the invention is predetermined by the ratio of any of the hydrophobic compound and the surfactant to the plant protein (as exemplified by FIG. 14 and further exemplified in the Examples section). In some embodiments, the size of the particle is controllable by adjusting the ratio of any of the hydrophobic compound and the surfactant to the plant protein.

In some embodiments, the size of the particle of the invention remains substantially the same after drying and reconstitution (as exemplified by FIG. 14). In some embodiments, upon drying and reconstitution at least 50%, at least 60%, at least 70%, at least 75%, at least 80%, at least 83%, at least 85%, at least 90%, at least 95% of the particles substantially retain a diameter thereof, wherein substantially is ±10%. As used herein, the diameter refers to the hydrodynamic particle diameter within a solution (or suspension).

In some embodiments, the nanoparticle of the invention significantly improves bioavailability or bio-accessibility of the hydrophobic compound.

### Micro Particles Containing Oil-Insoluble Hydrophobic Compound

In another aspect, there is a particle comprising a plant oil encapsulated in an amphiphilic shell, wherein the amphiphilic shell comprises an amphiphilic plant protein and a hydrophobic compound, wherein the w/w ratio of the hydrophobic compound to the plant protein ranges from 0.01:1 to 1:1, and wherein the w/w ratio of the plant protein to the plant oil ranges from 0.1:1 to 10:1.

In some embodiments, the particle comprises an oil-insoluble hydrophobic compound (e.g. curcuminoid), the plant oil, and the plant protein. In some embodiments, the particle is a micro-particle. In some embodiments, the particle is a core-shell particle. In some embodiments, the particle comprises a curcuminoid (e.g. curcumin), olive oil and PP, wherein the w/w ratios between the components within the particle are as described herein. In some embodiments, the particle core comprises the plant oil. In some embodiments, the particle shell (or the amphiphilic shell) comprises the plant protein and the oil-insoluble hydrophobic compound. In some embodiments, the particle comprises the amphiphilic shell encapsulating the particle core. In some embodiments, the amphiphilic shell is in contact with or bound to the particle core.

In some embodiments, the amphiphilic shell is a single layer shell. In some embodiments, the amphiphilic shell is in a form of a uniform layer. In some embodiments, the single layer amphiphilic shell comprises the oil-insoluble hydrophobic compound and the plant protein. In some embodiments, the oil-insoluble hydrophobic compound and the plant protein form a substantially homogenous single layer shell, wherein substantially is as described herein.

In some embodiments, the amphiphilic shell comprises a plurality of layers. In some embodiments, the amphiphilic shell comprises an inner portion or an inner layer facing the pant oil, and an outer portion or an outer layer facing the ambient. In some embodiments, the inner portion comprises the oil-insoluble hydrophobic compound and the plant protein. In some embodiments, the inner portion comprises the oil-insoluble hydrophobic compound (e.g. curcuminoid) bound to the plant protein (e.g. PP). In some embodiments, at least a part of the oil-insoluble hydrophobic compound is homogenously distributed within the amphiphilic shell. In some embodiments, “bound” is by a non-covalent bond or by a physical interaction. Such non-covalent bonds (e.g. hydrogen bonds, Van-der-Waals bonds) or other physical interactions (such as physisorption) are well-known in the art. In some embodiments, the outer portion of the amphiphilic shell comprises the plant protein (e.g. PP). In some embodiments, the outer portion of the amphiphilic shell substantially comprises the plant protein (e.g. PP). In some embodiments, the outer portion of the amphiphilic shell is substantially devoid of the oil-insoluble hydrophobic compound. In some embodiments, substantially is as described herein.

In some embodiments, at least a part of the oil-insoluble hydrophobic compound and of the plant protein form the amphiphilic shell, wherein the amphiphilic shell comprises a plurality of layers. In some embodiments, at least a part of the oil-insoluble hydrophobic compound and of the plant protein form the amphiphilic shell, wherein the amphiphilic shell comprises an inner portion and an outer portion as described herein.

In some embodiments, the amphiphilic shell comprising the oil-insoluble hydrophobic compound and the plant protein is in contact with or bound to the particle core comprising the plant oil. In some embodiments, the amphiphilic shell comprising the oil-insoluble hydrophobic compound and the plant protein encapsulates the plant oil. In some embodiments, the amphiphilic shell encapsulates the particle core comprising the plant oil and the oil-insoluble hydrophobic compound.

In some embodiments, at least 30%, at least 40%, at least 50%, at least 60%, at least 65%, at least 70%, at least 75%, at least 80%, at least 85%, at least 90%, at least 92%, at least 95%, at least 97%, at least 99% w/w of the oil-insoluble hydrophobic compound is incorporated within or bound to the inner portion of the amphiphilic shell.

In some embodiments, at most 30%, at most 40%, at most 50%, at most 60%, at most 65%, at most 70%, at most 75%, at most 80%, at most 85%, at most 90%, at most 92%, at most 95%, at most 97%, at most 99% w/w of the oil-insoluble hydrophobic compound is incorporated within or bound to the inner portion of the amphiphilic shell.

In some embodiments, at least 1%, at least 5%, at least 10%, at least 15%, at least 20%, at least 25%, at least 30%, at least 40%, at least 50% w/w of the oil-insoluble hydrophobic compound is incorporated (i.e. dissolved or dispersed) within the particle core (e.g. plant oil).

In some embodiments, at most 1%, at most 5%, at most 10%, at most 15%, at most 20%, at most 25%, at most 30%, at most 40%, at most 50% w/w of the oil-insoluble hydrophobic compound is incorporated (i.e. dissolved or dispersed) within the particle core (e.g. plant oil).

In some embodiments, at least 10%, at least 15%, at least 20%, at least 25%, at least 30%, at least 40%, at least 50%, at least 60%, at least 70%, at least 80%, at least 90%, at least 95%, at least 97%, at least 99%, w/w of the plant protein is incorporated within the outer portion of the amphiphilic shell.

In some embodiments, at most 10%, at most 15%, at most 20%, at most 25%, at most 30%, at most 40%, at most 50%, at most 60%, at most 70%, at most 80%, at most 90%, at most 95%, at most 97%, at most 99 w/w of the plant protein is incorporated within the outer portion of the amphiphilic shell.

In some embodiments, the amphiphilic shell substantially comprises the plant protein and the oil-insoluble hydrophobic compound of the invention, wherein substantially is as described herein.

In some embodiments, the w/w ratio of the hydrophobic compound to the plant oil in the particle ranges from 0.01:1 to 1:1. In some embodiments, the w/w ratio of the hydrophobic compound to the plant oil in the particle ranges from 0.01:1 to 0.05:1. In some embodiments, the w/w ratio of the hydrophobic compound to the plant oil in the particle ranges from 0.05:1 to 0.1:1. In some embodiments, the w/w ratio of the hydrophobic compound to the plant oil in the particle ranges from 0.1:1 to 0.2:1. In some embodiments, the w/w ratio of the hydrophobic compound to the plant oil in the particle ranges from 0.2:1 to 0.3:1. In some embodiments, the w/w ratio of the hydrophobic compound to the plant oil in the particle ranges from 0.3:1 to 1:1.

In some embodiments, the w/w ratio of the oil-insoluble hydrophobic compound (e.g. curcumin) to the plant oil (e.g. olive oil) within the particle is between 0.01:1 and 1:1, between 0.01:1 and 0.1:1, between 0.1:1 and 0.3:1, between 0.3:1 and 0.5:1, between 0.5:1 and 0.7:1, between 0.7:1 and 0.8:1, between 0.8:1 and 1:1, including any range or value therebetween.

In some embodiments the w/w ratio of the plant protein to the plant oil in the particle ranges from 0.1:1 to 10:1. In some embodiments the w/w ratio of the plant protein to the plant oil in the particle ranges from 1:1 to 4:1. In some embodiments the w/w ratio of the plant protein to the plant oil in the particle ranges from 0.1:1 to 0.5:1. In some embodiments the w/w ratio of the plant protein to the plant oil in the particle ranges from 0.5:1 to 1:1. In some embodiments the w/w ratio of the plant protein to the plant oil in the particle ranges from 1.5:1 to 2:1. In some embodiments, the w/w ratio of the plant protein to the plant oil in the particle ranges from 0.5:1 to 1:1. In some embodiments, the w/w ratio of the plant protein to the plant oil in the particle ranges from 1:1 to 2:1. In some embodiments, the w/w ratio of the plant protein to the plant oil in the particle ranges from 2:1 to 5:1. In some embodiments, the w/w ratio of the plant protein to the plant oil in the particle ranges from 5:1 to 10:1. In some embodiments, the w/w ratio of the plant protein to the plant oil in the particle ranges from 10:1 to 15:1.

In some embodiments the w/w ratio of the plant protein (e.g. PP) to the plant oil (e.g. olive oil) within the particle is between 0.1:1 and 10:1, between 0.1:1 and 0.5:1, between 0.5:1 and 1:1, between 1:1 and 2:1, between 2:1 and 3:1, between 3:1 and 4:1, between 4:1 and 5:1, between 5:1 and 6:1, between 6:1 and 8:1, between 8:1 and 10:1, including any range or value therebetween.

In some embodiments, the emulsion comprises the hydrophobic compound at the concentration of 1 to 10000 ppm. In some embodiments, the emulsion comprises the hydrophobic compound at the concentration of 1 to 5000 ppm. In some embodiments, the emulsion comprises the hydrophobic compound at the concentration of 1 to 3000 ppm. In some embodiments, the emulsion comprises the hydrophobic compound at the concentration of 1 to 2000 ppm. In some embodiments, the emulsion comprises the hydrophobic compound at the concentration of 1 to 1000 ppm. In some embodiments, the emulsion comprises the hydrophobic compound at the concentration of 100 to 1000 ppm. In some embodiments, the emulsion comprises the hydrophobic compound at the concentration of 300 to 1000 ppm. In some embodiments, the emulsion comprises the hydrophobic compound at the concentration of 300 to 2000 ppm. In some embodiments, the emulsion comprises the hydrophobic compound at the concentration of 500 to 3000 ppm. In some embodiments, the emulsion comprises curcumin at a concentration of 5 to 5000 ppm. In some embodiments, the dried emulsion powder comprises curcumin at a concentration of 50 to 500 ppm.

In some embodiments, the composition (e.g., in a form of a powder) comprises the hydrophobic compound at the concentration of 100 to 1000000 ppm, 100 to 100000 ppm, or 100 to 10000 ppm.

In some embodiments, the particle comprises the hydrophobic oil-insoluble compound at the concentration of 100 to 1,000,000 ppm, of 100 to 1,000 ppm, of 1,000 to 10,000 ppm, of 1,000 to 2,000 ppm, of 2,000 to 4,000 ppm, of 4,000 to 6,000 ppm, of 6,000 to 8,000 ppm, of 8,000 to 10,000 ppm, of 10,000 to 12,000 ppm, of 12,000 to 15,000 ppm, of 15,000 to 20,000 ppm, of 20,000 to 30,000 ppm, of 30,000 to 40,000 ppm, of 40,000 to 50,000 ppm, of 50,000 to 100,000 ppm, of 100,000 to 200,000 ppm, of 200,000 to 500,000 ppm, of 500,000 to 1000,000 ppm, including any range or value therebetween.

In some embodiments, the particle comprises the hydrophobic oil-insoluble compound at the concentration of at most 100,000 ppm, at most 90,000 ppm, at most 80,000 ppm, at most 70,000 ppm, at most 60,000 ppm, at most 50,000 ppm, at most 40,000 ppm, at most 30,000 ppm, at most 20,000 ppm, at most 10,000 ppm, at most 8,000 ppm, at most 5,000 ppm, at most 2,000 ppm, at most 1,000 ppm, at most 800 ppm, at most 500 ppm, at most 100 ppm, including any range or value therebetween.

In some embodiments, the particle size of the emulsion ranges from 0.015 to 50 μm. In some embodiments, the particle size ranges from 0.015 to 0.5 μm. In some embodiments, the particle size of the emulsion ranges from 0.1 to 10 μm. In some embodiments, the particle size of the emulsion ranges from 0.2 to 3 μm. In some embodiments, the particle size of the emulsion ranges from 1 to 5 μm. In some embodiments, the particle size of the emulsion ranges from 5 to 8 μm. In some embodiments, the particle size of the emulsion ranges from 7 to 10 μm. In some embodiments, the particle size of the emulsion ranges from 10 to 30 μm. In some embodiments, the particle size of the emulsion ranges from 2 to 30 μm. In some embodiments, the particle size is as described herein.

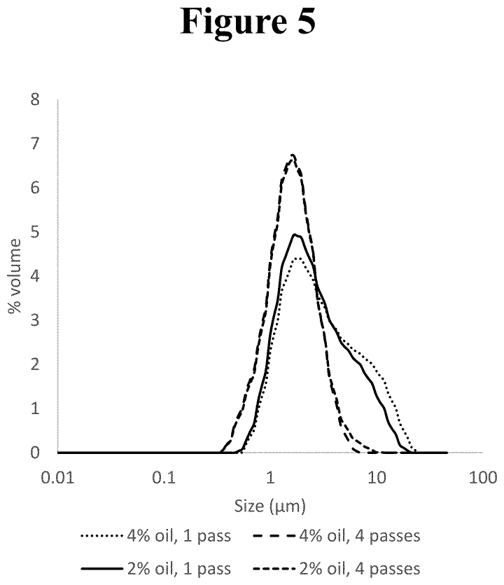

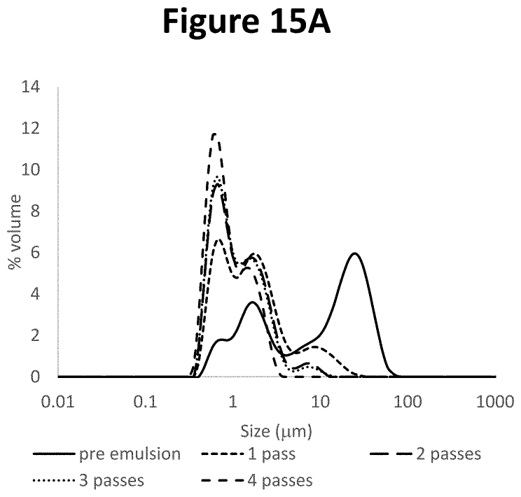

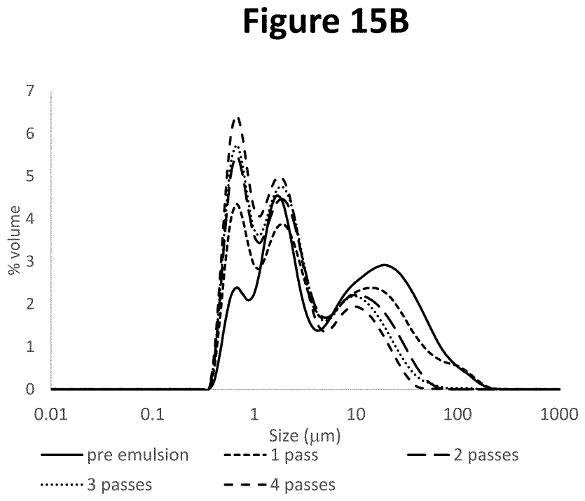

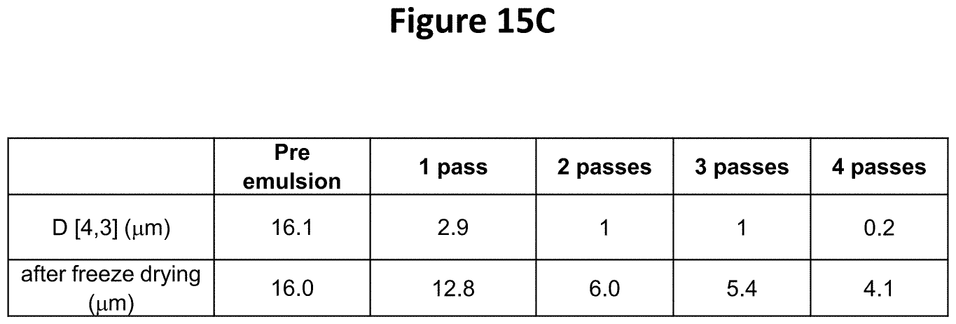

In some embodiments, the particle size is predetermined by a number of the homogenization steps, wherein homogenization is as described herein. As exemplified by FIGS. 5 and 15, the particle size decreases by performing a plurality of homogenization steps.

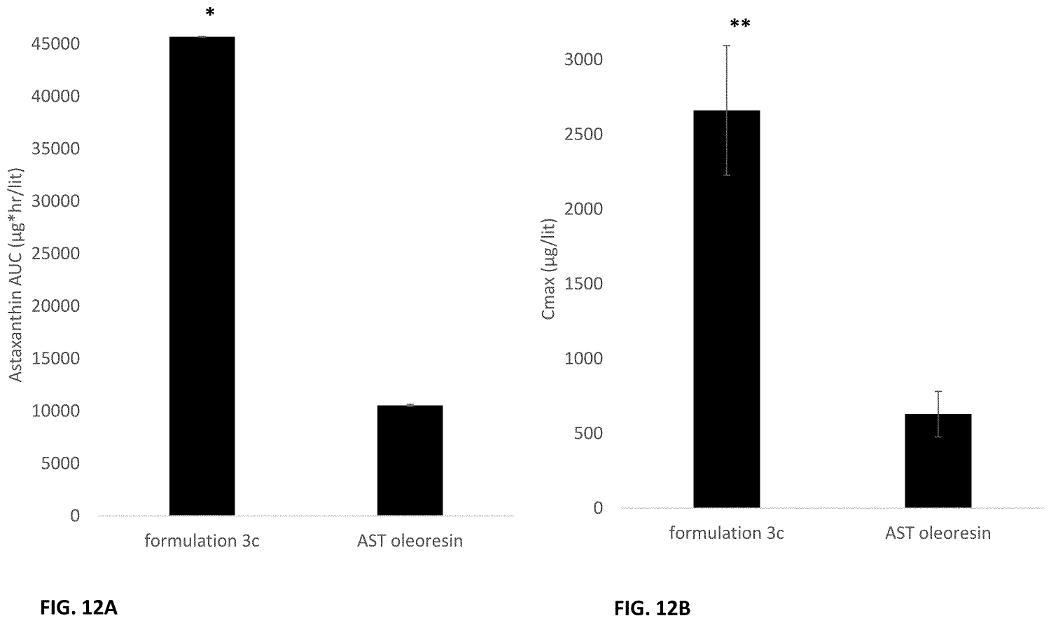

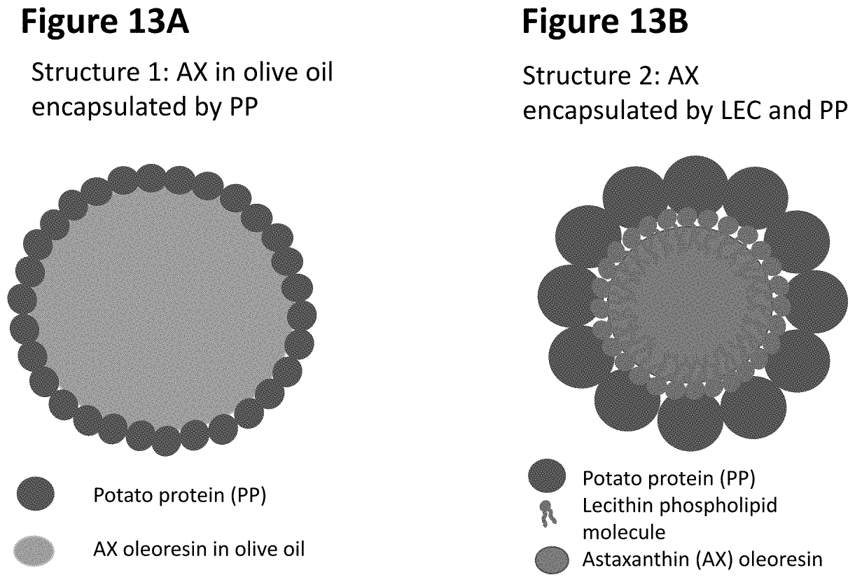

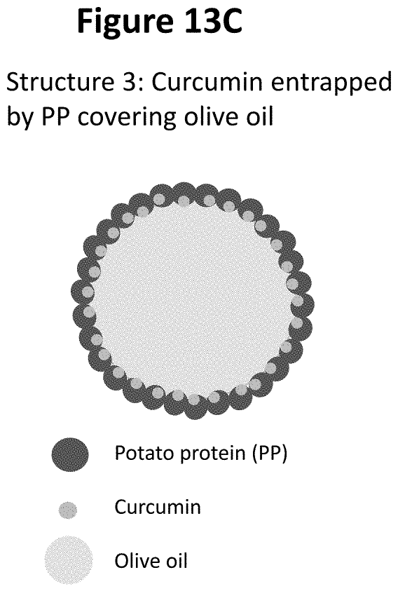

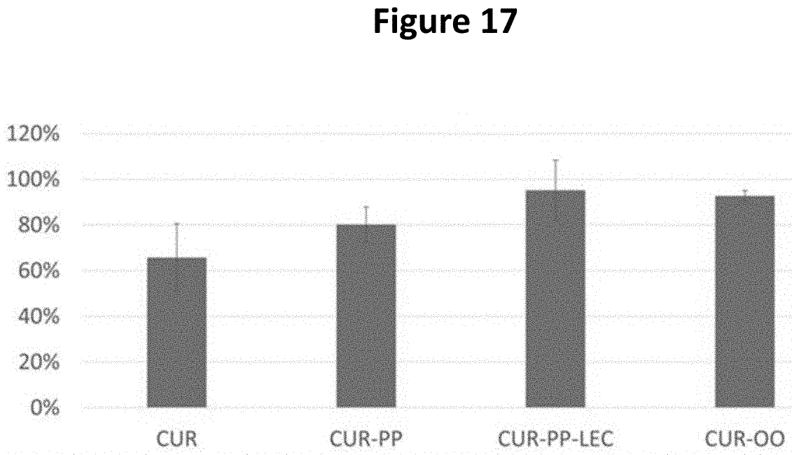

In some embodiments, the particle of the invention is characterized by an improved bioaccessibility of the hydrophobic compound (as exemplified by FIG. 12). In some embodiments, the bioaccessibility of the hydrophobic compound encapsulated by the particle of the invention is enhanced by at least 10%, at least 50%, at least 70%, at least 100%, at least 150%, at least 200%, at least 300%, at least 500%, at least 400%, at least 600%, at least 800%, at least 1000% as compared to the bioaccessibility of the non-encapsulated hydrophobic compound. In some embodiments, the particle of the invention is characterized by an increased protection of the encapsulated curcumin, as compared to a non-encapsulated curcumin, or to curcumin encapsulated by PP (FIG. 17).

### Dry Formulation

In some embodiments, the composition of the present invention is a powderous composition comprising the hydrophobic compound. In some embodiments, the powderous composition comprises the particle. In some embodiments, the amphiphilic shell comprising the plant protein encapsulates the hydrophobic core comprising the plant oil. In some embodiments, the powderous composition comprises the plant protein, the plant oil, and the hydrophobic compound. In some embodiments, the powderous composition comprises the plant protein, the surfactant, and the hydrophobic compound. In some embodiments, the powderous composition comprises the oil-insoluble hydrophobic compound bound to the plant protein, and the plant oil. In some embodiments, the powderous composition comprises the ethanol soluble hydrophobic compound bound to the plant protein, and the plant oil.

In some embodiments, the powderous composition further comprises a cryoprotectant. In some embodiments, the cryoprotectant is tapioca maltodextrin. In some embodiments, the powderous composition is a dry formulation.

In some embodiments, the water content of the dry formulation is less than 10% w/w. In some embodiments, the water content of the dry formulation is less than 5% w/w. In some embodiments, the water content of the dry formulation is less than 2% w/w. In some embodiments, the water content of the dry formulation is less than 1% w/w.

### Reconstituted Formulation

In some embodiments, the dry formulation forms a stable aqueous formulation upon reconstitution with water. In some embodiments, the dry formulation forms a stable emulsion upon reconstitution with water. In some embodiments, the dry formulation forms a stable oil-in-water emulsion upon reconstitution. In some embodiments, the reconstituted oil-in-water emulsion comprises the hydrophobic compound, the plant oil, and the plant protein. In some embodiments, the reconstituted oil-in-water emulsion comprises the ethanol soluble hydrophobic compound, the plant oil, and the plant protein. In some embodiments, the reconstituted oil-in-water emulsion comprises the hydrophobic compound, the plant protein and the surfactant. In some embodiments, the w/w concentration of the hydrophobic compound in the reconstituted emulsion ranges from 1 to 10000 ppm.

In some embodiments, the reconstituted emulsion comprises the particle dispersed in an aqueous solution. In some embodiments, the particle size of the reconstituted emulsion ranges from 0.1 to 100 μm. In some embodiments, the particle size of the reconstituted emulsion ranges from 0.1 to 1 μm. In some embodiments, the particle size of the reconstituted emulsion ranges from 0.1 to 2 μm. In some embodiments, the particle size of the reconstituted emulsion ranges from 0.3 to 3 μm. In some embodiments, the particle size of the reconstituted emulsion ranges from 1 to 4 μm. In some embodiments, the particle size of the reconstituted emulsion ranges from 1 to 10 μm. In some embodiments, the particle size of the reconstituted emulsion ranges from 1 to 30 μm. In some embodiments, the particle size of the reconstituted emulsion ranges from 1 to 50 μm. In some embodiments, the particle size of the reconstituted emulsion ranges from 10 to 30 μm. In some embodiments, the particle size of the reconstituted emulsion ranges from 30 to 50 μm.

In some embodiments, the reconstituted emulsion releases the hydrophobic compound (e.g. AX) upon simulated digestion. In some embodiments, the reconstituted emulsion releases more than 30 wt % of the initial content of the hydrophobic compound. In some embodiments, the reconstituted emulsion releases more than 40 wt % of the initial content of the hydrophobic compound. In some embodiments, the reconstituted emulsion releases more than 50 wt % of the initial content of the hydrophobic compound. In some embodiments, the reconstituted emulsion releases more than 60 wt % of the initial content of the hydrophobic compound. In some embodiments, the reconstituted emulsion releases more than 70 wt % of the initial content of the hydrophobic compound. In some embodiments, the reconstituted emulsion releases more than 80 wt % of the initial content of the hydrophobic compound. In some embodiments, the reconstituted emulsion releases more than 90 wt % of the initial content of the hydrophobic compound. Detailed description of the simulated digestion and a method for estimating free hydrophobic compound content, is provided in the experimental section.

### Method of Preparation (Emulsion)

In some embodiments, the present invention comprises a method for solubilizing the hydrophobic compound in an aqueous solution. In some embodiments, the present invention comprises a method for solubilizing the hydrophobic compound in an aqueous formulation. In some embodiments, the present invention comprises a method for solubilizing the hydrophobic compound in an oil-in-water emulsion. In some embodiments, the method for solubilizing the hydrophobic compound in the aqueous solution comprises producing the oil-in-water emulsion, as described in method A.

In some embodiments, method A for comprises the following steps:

a) producing a pre-mix by mixing at least one hydrophobic compound in the plant oil at the temperature from 30 to 70° C., wherein the final w/w concentration of the hydrophobic compound in pre-mix is in the range from 0.1 to 30%;

b) producing a solution of the plant protein by dissolving the plant protein in an aqueous phase, to obtain a final w/w concentration of the plant protein in the solution ranging from 1 to 20%;

c) introducing the solution of step b) into the pre-mix of step a);

d) performing at least one step of pre-homogenization by processing the mixture of step c) using a rotator mixer at the speed ranging from 1,000 rpm to 100,000 rpm, from 1 to 30 min;

e) performing at least one step, of high-pressure homogenization at the pressure ranging from 300 to 3000 bar.

In some embodiments, the final w/w concentration of the hydrophobic compound in the pre-mix of step a) is in the range from 0.1 to 4%. In some embodiments, the final w/w concentration of the hydrophobic compound in the pre-mix of step a) is in the range from 0.1 to 1%, from 1 to 4%, from 2 to 4%.

In some embodiments, the step a) is performed at 50° C. for 30 min or until the mixture is obtained at a homogenous condition.

In some embodiments, the solution of step b) comprises the plant protein at the w/w concentration from 0.1 to 10%. In some embodiments, the solution of step b) comprises the plant protein at the w/w concentration from 0.1 to 1%, from 1 to 4%, from 4 to 10, from 3 to 5%.

In some embodiments, the solution of step b) comprises water. In some embodiments, the solution of step b) comprises an aqueous buffer solution. In some embodiments, the solution of step b) comprises a phosphate buffer. In some embodiments, the pH of the solution ranges from 4 to 8. In some embodiments, the pH of the solution is ranges from 6.5 to 7.5. In some embodiments, the pH of the solution is ranges from 2 to 6.

In some embodiments, the solution of step b) comprises the plant protein and a cryoprotectant. In some embodiments, the w/w concentration of the cryoprotectant in the solution of step b) ranges from 0.5 to 10%. In some embodiments, the w/w concentration of the cryoprotectant in the solution of step b) ranges from 0.5 to 2%, from 2 to 4%, from 4 to 6%, from 6 to 10%.

In some embodiments, the cryoprotectant is tapioca maltodextrin.

In some embodiments, the pre-homogenization of step d) is performed at the speed ranging from 1,000 to 50,000 rpm. In some embodiments, the pre-homogenization of step d) is performed at the speed ranging from 10,000 to 35,000 rpm. In some embodiments, the pre-homogenization of step d) is performed from 1 to 30 min.

In another aspect of the invention, there is a method for producing the oil-in-water emulsion comprising the steps a) to c), and further comprising the step e). In some embodiments, the step e) is performed at the pressure ranging from 300 to 3000 bar.

In some embodiments, the step e) is performed one or more times.

In some embodiments, the present invention comprises a method for solubilizing the hydrophobic compound in an oil in water emulsion by using a surfactant, as described in method B.

In some embodiments, method B comprises the following steps:

a) producing an ethanolic solution by mixing the surfactant and the hydrophobic compound with ethanol to obtain a final molar ratio of the hydrophobic compound to the surfactant in the solution ranging from 0.01:1 to 2:1;

b) producing a solution of the plant protein by dissolving the plant protein in an aqueous phase, to obtain a final concentration of the plant protein in the solution ranging from 1 to 20%;

c) introducing the solution of step a) into the solution of step b);

d) performing at least one step of pre-homogenization and at least one step, of high-pressure homogenization, as described herein.

In some embodiments, the solution of step b) comprises the plant protein at the w/w concentration from 0.1 to 10%. In some embodiments, the solution of step b) comprises the plant protein at the w/w concentration from 0.1 to 1%, from 1 to 4%, from 4 to 10, from 3 to 5%.

In some embodiments, the solution of step b) comprises water. In some embodiments, the solution of step b) comprises an aqueous buffer solution. In some embodiments, the solution of step b) comprises a phosphate buffer. In some embodiments, the pH of the solution ranges from 4 to 8. In some embodiments, the pH of the solution is ranges from 6.5 to 7.5. In some embodiments, the pH of the solution is ranges from 2 to 6.

In some embodiments, the concentration of the hydrophobic compound in the ethanolic solution of step a) is in the range from 0.1 to 50 mM.

In some embodiments, the concentration of the surfactant in the ethanolic solution of step a) is in the range from 0.1 to 50 mM.

In some embodiments, the pre-homogenization of step d) is performed at the speed ranging from 1,000 to 50,000 rpm. In some embodiments, the pre-homogenization of step d) is performed at the speed ranging from 10,000 to 35,000 rpm. In some embodiments, the pre-homogenization of step d) is performed from 1 to 30 min.

In some embodiments, the present invention comprises a method for solubilizing the ethanol soluble hydrophobic compound in an oil in water emulsion as described in method C. In some embodiments, method C comprises the following steps:

a) producing an ethanolic solution by mixing the hydrophobic compound with ethanol to obtain a final molar ratio of the hydrophobic compound to the surfactant in the solution ranging from 0.01:1 to 2:1;

b) producing a solution of the plant protein by dissolving the plant protein in an aqueous phase, to obtain a final concentration of the plant protein in the solution ranging from 1 to 20%;

c) introducing the solution of step a) into the solution of step b) to obtain a combined aqueous solution;

d) mixing the aqueous solution with a plant oil to form a mixture;

e) performing at least one step of pre-homogenization and at least one step, of high-pressure homogenization, as described herein.

### Method of Preparation (Powderous Formulation)

In some embodiments, provided herein a method for producing a powderous composition. In some embodiments, a method for producing the powderous composition comprises any one of the methods A to C for producing the oil-in-water emulsion. In some embodiments, a method for producing the powderous composition further comprises the step f) of drying the oil-in-water emulsion, to obtain a dry powder.

In some embodiments, the step f) comprises lyophilization. In some embodiments, the step f) comprises freeze drying. In some embodiments, the step f) comprises spray drying.

In some embodiments, the water content of the dry powder is less than 10% w/w. In some embodiments, the water content of the dry powder is less than 5% w/w. In some embodiments, the water content of the dry powder is less than 2% w/w. In some embodiments, the water content of the dry powder is less than 1% w/w.

In some embodiments, provided herein a method for producing a capsule, comprising a hydrophobic compound as a dietary supplement.

In some embodiments, the method for producing the capsule comprises: the method for producing the powderous composition, to obtain a dry powder; and adding the dry powder into a capsule.

In some embodiments, the amount of dry powder in the capsule ranges from 10 to 2000 mg. In some embodiments, the amount of dry powder in the capsule ranges from 20 to 1000 mg. In some embodiments, the amount of dry powder in the capsule ranges from 40 to 500 mg. In some embodiments, the amount of dry powder in the capsule ranges from 60 to 100 mg. In some embodiments, the amount of dry powder in the capsule ranges from 20 to 100 mg.

### Methods of Use

In another aspect, the present invention is directed to a method for enhancing bioaccessibility of a hydrophobic compound. In some embodiments, the method for enhancing bioaccessibility of a hydrophobic compound comprises administering the composition (e.g. the particle, the emulsion or the formulation) of the invention to a subject in need thereof, thereby increasing bioaccessibility of the hydrophobic compound within the subject. In some embodiments, the method for enhancing bioavailability of a hydrophobic compound comprises administering the composition of the invention to a subject in need thereof, thereby increasing bioavailability of the hydrophobic compound within the subject. In some embodiments, the composition comprises any of the particles of the invention. In some embodiments, the hydrophobic compound is as described herein. The term “bioaccessibility” as used herein, is directed to ability to release a hydrophobic compound in-vitro by any one of the formulations of the present invention. The in-vitro release can be evaluated by using a protocol of simulated digestion. The protocol is provided on the experimental section. The bioaccessible fraction is the fraction of the active hydrophobic compound found in the supernatant following simulated gastrointestinal digestion and centrifugation.

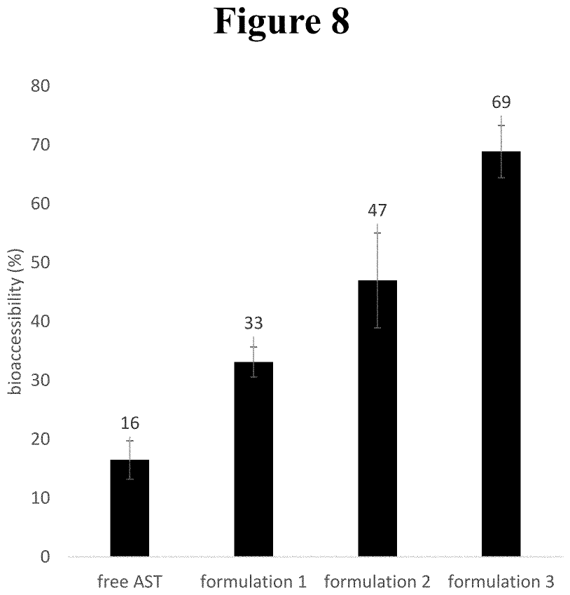

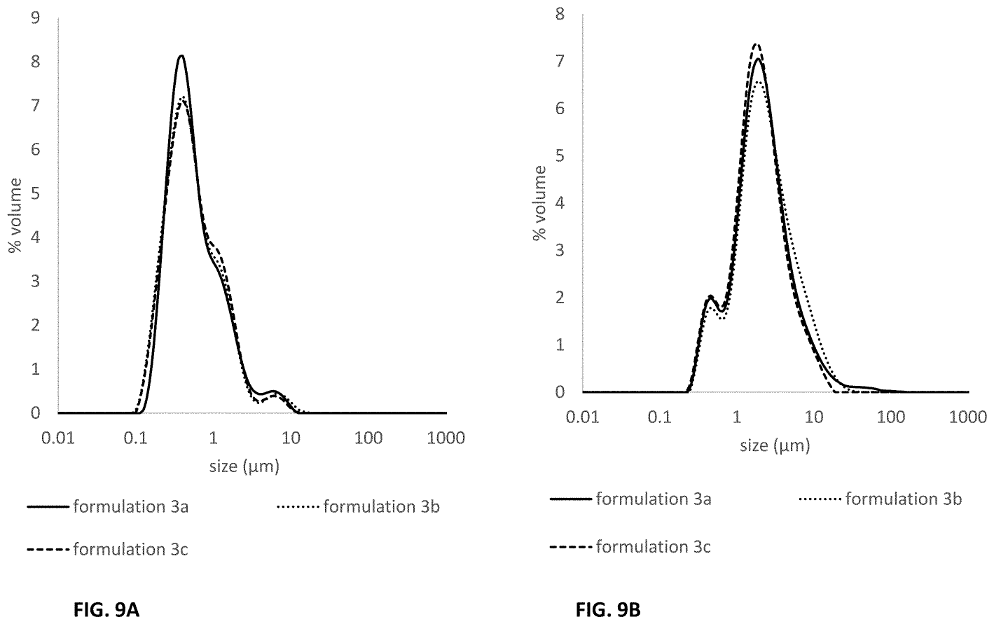

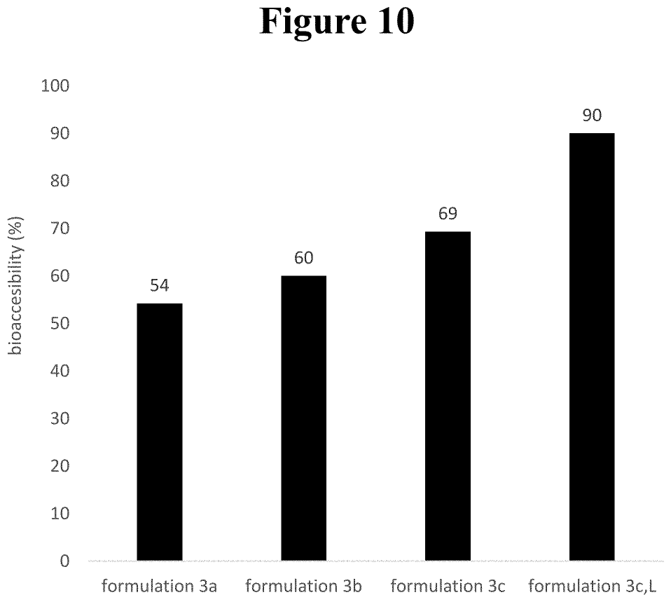

In some embodiments, the composition of the present invention enhances the bioaccessibility of a hydrophobic compound. In some embodiments, the composition of the present invention enhances the bioaccessibility of the oil-soluble hydrophobic compound within a subject, wherein the oil-soluble hydrophobic compound is as described herein (such as a carotenoid). In some embodiments, the composition of the present invention enhances the bioaccessibility of the oil-insoluble hydrophobic compound within a subject, wherein the oil-insoluble hydrophobic compound is as described herein (such as a curcuminoid). In some embodiments, the formulation of the present invention provides an enhancement of AX bioaccessibility, as compared to AX oleoresin. In some embodiments, the formulation is an oil-in-water (o/w) emulsion comprising AX. In some embodiments, the bioaccessibility of formulated AX is increased by a factor of 4.3, as compared to AX oleoresin (FIG. 8). In some embodiments, the bioaccessibility of encapsulated curcumin is increased by a factor of about 2, as compared to non-encapsulated curcumin.

In some embodiments, increasing or enhancing is by at least 50%, at least 70%, at least 100%, at least 150%, at least 200%, at least 300%, at least 500%, at least 400%, at least 450%, at least 500%, at least 550%, at least 600%, at least 700%, at least 800%, at least 900%, at least 1000% as compared to the bioaccessibility of the non-encapsulated hydrophobic compound.

In some embodiments, the present invention is directed to a method for enhancing bioavailability of a hydrophobic compound. In some embodiments, the method comprises administering a capsule, comprising the hydrophobic compound to a subject in need thereof. In some embodiments, the method comprises administering the composition of the present invention to a subject in need thereof. In some embodiments, the method comprises administering the reconstituted composition of the present invention to a subject in need thereof. In some embodiments, the composition is o/w emulsion. In some embodiments, the composition is a powderous composition.

In some embodiments, the present invention is directed to a method for enhancing the concentration of a hydrophobic compound in blood plasma in a subject in need thereof. In some embodiments, the method comprises administering the composition of the present invention to a subject. In some embodiments, the method comprises administering the capsule, comprising the hydrophobic compound to a subject.

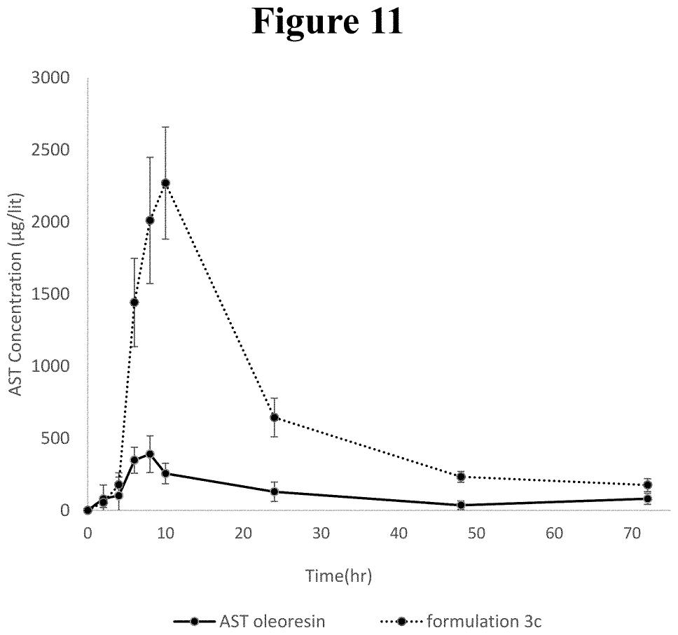

In some embodiments, provided herein is a method for enhancing the concentration of the hydrophobic compound (e.g. AX and/or curcumin) in blood plasma in a subject in need thereof. In some embodiments, the method comprises administering to a subject the composition of the present invention. In some embodiments, the maximal plasma concentration (Cmax) measured after administering the composition to a subject is increased by a factor of 4.5, as compared to non-encapsulated hydrophobic compound (e.g. AX oleoresin) (FIG. 11). In some embodiments, the Cmax measured after administering the composition to a subject, ranges from 0.2 mg/L to 25 mg/L. In some embodiments, a total plasma concentration measured over 72 h after administering the composition a subject, is increased by a factor of 4.5, as compared to AX oleoresin (FIG. 12).

In some embodiments, there is a method of supplementing a subject with a hydrophobic compound, comprising the step of administering to a subject the composition of the present invention. In some embodiments, the method comprises administering to a subject the reconstituted composition of the present invention. In some embodiments, the composition further comprises an aqueous liquid. In some embodiments, an aqueous liquid comprises the hydrophobic compound formulated within the composition. In some embodiments, an aqueous liquid comprises oil-in-water emulsion comprising the hydrophobic compound. In some embodiments, the method of supplementing a subject with the hydrophobic compound comprises administering to a subject the capsule comprising the hydrophobic compound.

In some embodiments, a subject is a human. In some embodiments, a subject is a pet. In some embodiments, a subject is a farm animal. In some embodiments, a subject is a rodent. In some embodiments, a subject is an infant. In some embodiments, a subject is a toddler.

In some embodiments, the present invention further provides a method of supplementing a subject with a nutraceutical as a hydrophobic compound, comprising the step of administering to a subject a capsule comprising the composition of the present invention. In some embodiments, the capsule is administered orally.

In some embodiments, a nutraceutical is any non-toxic food component which has demonstrated health benefits. In some embodiments, a nutraceutical is any sparingly water soluble, non-toxic food component, which has demonstrated health benefits. In some embodiments, a nutraceutical is any fat soluble, non-toxic food component, which has demonstrated health benefits.

Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of a subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### Pharmaceutical Compositions

In some embodiments, the composition of the present invention comprises a pharmaceutical composition. In some embodiments, the pharmaceutical composition is presented in form of a liquid formulation (e.g. oil-in-water emulsion). In some embodiments, the pharmaceutical composition is presented in form of a powder. In some embodiments, the pharmaceutical composition is presented in form of a capsule comprising a liquid formulation. In some embodiments, the pharmaceutical composition is presented in form of a capsule comprising a liquid concentrate. In some embodiments, the pharmaceutical composition is presented in form of a capsule comprising a powder.

In some embodiments, the capsule comprises 5 to 100 mg of the hydrophobic compound. In some embodiments, the capsule comprises 5 to 20 mg of the hydrophobic compound. In some embodiments, the capsule comprises 10 to 20 mg of the hydrophobic compound. In some embodiments, the capsule comprises 20 to 40 mg of the hydrophobic compound. In some embodiments, the capsule comprises 40 to 100 mg of the hydrophobic compound.

In some embodiments, the capsule comprises from 6 to 20 mg of the hydrophobic compound, corresponding to 10-300% of recommended daily uptake.

In some embodiments, the capsule further comprises other food additives. Non-limiting examples of food additives include but are not limited to: flavonoids, carnitine, choline, vitamins, hydrophobic vitamins, polyunsaturated fatty acids, coenzyme Q, creatine, dithiolthiones, phytosterols, polysaccharides, nutraceuticals, antioxidants, phytoestrogens, glucosinolates, polyphenols, anthocyanins, or any combination thereof.

In some embodiments, the hydrophobic compound is administered at a dosage of 6-600 mg/day. In some embodiments, the hydrophobic compound is administered at a dosage of 50-100 mg/day. In some embodiments, the hydrophobic compound is administered at a dosage of 100-200 mg/day. In some embodiments, the hydrophobic compound is administered at a dosage of 200-400 mg/day. In some embodiments, the hydrophobic compound is administered at a dosage of 6-20 mg/day. In some embodiments, the hydrophobic compound is administered at a dosage of 40-70 mg/day.

In some embodiments, the hydrophobic compound is present at a concentration of at least 0.01 mg/ml, at least 0.1 mg/ml, at least 0.5 mg/ml, at least 1 mg/ml, at least 5 mg/ml, at least 10 mg/ml, at least 15 mg/ml, at least 20 mg/ml, at least 25 mg/ml, at least 30 mg/ml, at least 35 mg/ml, at least 40 mg/ml, at least 50 mg/ml, at least 60 mg/ml, at least 70 mg/ml, at least 80 mg/ml, at least 90 mg/ml, at least 100 mg/ml, at least 200 mg/ml, or any range therebetween, within the composition. In some embodiments, the hydrophobic compound is present at a concentration of 0.1-1 mg/ml, 0.05-1.5 mg/ml, 1-5 mg/ml, 4-10 mg/ml, 6-12 mg/ml, 11-15 mg/ml, 12-20 mg/ml, 15-25 mg/ml, 20-35 mg/ml, 30-45 mg/ml, 40-60 mg/ml, 50-70 mg/ml, 60-80 mg/ml, 70-90 mg/ml, or 80-100 mg/ml within the composition. Each possibility represents a separate embodiment of the invention.

An embodiment of the invention relates to any composition of the present invention comprising at least one hydrophobic compound and/or at least one food additive, presented in unit dosage form and prepared by any of the methods well known in the art of pharmacy. In some embodiments, the unit dosage form is in the form of a tablet, capsule, lozenge, wafer, patch, ampoule, vial or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

As used herein, the terms “administering”, “administration”, and like terms refer to any method which, in sound medical practice, delivers a composition containing an active agent to a subject in such a manner as to provide a therapeutic effect.

For oral applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type an excipient. In addition, dosage unit forms can contain various other materials which modify the physical form of the dosage unit, for example, coatings of sugar, shellac, or other enteric agents. The tablets of the invention can further be film coated. In some embodiment, oral application of the pharmaceutical composition may be in the form of drinkable liquid. In some embodiment, oral application of the pharmaceutical composition may be in the form of an edible product.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. Suitable capsules include pullulan or gelatin capsules or any other capsules allowed for use in dietary supplements or pharmaceuticals. The composition, if desired, can also contain minor amounts of pH buffering agents such as acetates, citrates or phosphates. Antibacterial agents such as benzyl alcohol or methyl parabens; antioxidants such as ascorbic acid or sodium bisulfate; and agents for the adjustment of tonicity such as sodium chloride are also envisioned.

In some embodiments, the present invention provides combined preparations. In some embodiments, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In some embodiments, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In some embodiments, depending on the severity and responsiveness of the condition to be treated, dosing can be of a single or a plurality of administrations, with course of treatment lasting from several days to several weeks or until cure is affected or diminution of the disease state is achieved.

In some embodiments, the composition of the preset invention is administered in a therapeutically safe and effective amount. As used herein, the term “safe and effective amount” refers to the quantity of a component which is sufficient to yield a desired therapeutic response without undue adverse side effects, including but not limited to toxicity, such as calcemic toxicity, irritation, or allergic response, commensurate with a reasonable benefit/risk ratio when used in the presently described manner.

In some embodiments, toxicity and therapeutic efficacy of the active ingredients described herein can be determined by standard pharmaceutical procedures in vitro, in cell cultures or experimental animals. In some embodiments, the data obtained from these in vitro and cell culture assays and animal studies can be used in formulating a range of dosage for use in human. In some embodiments, the dosages vary depending upon the dosage form employed and the route of administration utilized. In some embodiments, the exact formulation, route of administration and dosage can be chosen by the individual physician in view of the patient's condition. [See e.g., Goodman and Gilman's The Pharmacological Basis of Therapeutics, 13^{th} Ed., McGraw-Hill/Education, New York, N.Y. (2017)].

In some embodiments, compositions including the preparation of the present invention formulated in a compatible pharmaceutical carrier are prepared, placed in an appropriate container, and labeled for treatment of an indicated condition.

In some embodiments, compositions of the present invention are presented in a pack or dispenser device, such as an FDA approved kit, which contains, one or more unit dosages forms containing the active ingredient. In some embodiments, the pack, for example, comprises metal or plastic foil, such as a blister pack. In some embodiments, the pack or dispenser device is accompanied by instructions for administration. In some embodiments, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in some embodiments, is labeling approved by the U.S. Food and Drug Administration for prescription drugs or of an approved product insert.

### EXAMPLES

Generally, the nomenclature used herein, and the laboratory procedures utilized in the present invention include molecular, biochemical, and microbiological techniques. Such techniques are thoroughly explained in the literature.

### Materials and Methods

### System Characterization

Particle size distribution of Astaxanthin-Potato Protein (AX-PP), Astaxanthin-Lecithin-Potato Protein (AX-LEC-PP), pure curcumin, CUR-PP and CUR-LEC-PP samples was determined using a NICOMP DLS analyzer. Particle size distribution of Astaxanthin-Olive oil-Potato Protein (AX-Olive oil-PP) emulsion and of CUR-Olive oil-PP emulsion was determined using a Malvern Mastersizer 3000. Statistical analysis of the results was performed using Microsoft EXCEL 2013. The samples were tested in duplicate.

Crystal formation and particles stability were studied by light microscopy, as ways to determine optimal loading capacity, using an Olympus DP71 digital camera connected to an Olympus BX51 light microscope, operated in a bright-field optical mode or in polarized light optics.

### Freeze Drying and Reconstitution

Samples were freeze dried using Labconco benchtop Freeze Dryer overnight and kept at −20° C. until analysis. Before simulated digestion or particle size analysis, the samples were reconstituted to the initial volume with distilled water and kept at−20° C. until simulated digestion or particle size analysis.

### Simulated Digestion

Simulated gastric and intestinal digestion was based on the protocol described by Minekus et al. Food Funct., 2014, 5, 1113-1124. To evaluate CUR protection, the digested samples underwent extraction and the CUR content was determined by spectrophotometer with λ=420 nm. To evaluate CUR bioaccessibility, the digested samples underwent ultra-centrifugation (15,000 rpm, 30 min., 20° C.), the upper liquid was collected and its CUR content was determined by extraction and spectrophotometry (absorbance at λ=420 nm). Extraction by phase separation was achieved by adding ethyl acetate and ethanol (2:1 v/v), followed by 1 min of vortex. 500 μl of water were then added to each test tube and vortexed for 20 sec. The samples were then centrifuged for 5 min at 5,000 G at 25° C. The upper layer of ethyl acetate containing CUR was removed and the steps of addition of 1 ml of ethyl acetate were repeated once more. The ethyl acetate was evaporated from the extract under a flow of nitrogen, and reconstituted in a known volume of ethanol.

### Example 1

### Encapsulation of Astaxanthin by Astaxanthin-Potato Protein Nanoparticles Formation

An ethanolic stock solution of Astaxanthin (AX) oleoresin was added dropwise into potato protein (PP) solution (in phosphate buffer, pH=7) to obtain two different molar ratios (AX in excess). The molar ratios obtained were 1:1 and 2:1 (AX:PP). The concentration of AX was 0.5 mM in all samples.

### Example 2

### Encapsulation of Astaxanthin by Astaxanthin-Lecithin-Potato Protein Nanoparticles Formation

A pre-mix of the AX stock solution with the ethanol-soluble fraction of sunflower lecithin (LEC) in ethanol was prepared (equal concentration of AX and LEC, 5 mM). The pre-mix was added dropwise into PP solution (in phosphate buffer, pH=7) to obtain different molar ratios (AX and LEC in excess). The molar ratios obtained were 1:1:1 and 2:2:1 (AX:LEC:PP). The concentration of AX was 0.5 mM in all samples.

### Example 3

### Encapsulation of Astaxanthin in Astaxanthin-Olive Oil-Potato Protein Emulsion

A pre-mix of AX oleoresin and refined olive oil (1:3 w/w AX Oleoresin:Olive oil) was left shaking in 50° C. for 30 min until the mixture was homogenous. PP solution (in phosphate buffer, pH=7) was added to the AX:Olive oil mixture. Two different proportion were tested—1:3:4 w/w AX Oleoresin:Olive oil:PP in the final solution (4% oil) or 0.5:1.5:4 w/w AX Oleoresin:Olive oil:PP in the final solution (2% oil). Then, the mixture was pre-homogenized using a desktop homogenizer (30,000 rpm, 5 min). The pre-emulsion was homogenized using a high-pressure homogenizer (Emulsiflex C3, Avesin, 950 bar, 4 passes).

### Example 4

### Encapsulation of Curcumin in Curcumin-PP-Olive Oil Emulsion

Curcumin was dissolved in ethanol. The ethanolic curcumin solution was then added into a PP solution while stirring. Olive oil was then added while prehomogenizing, using a desktop homogenizer (30,000 rpm, 5 min). The pre-emulsion was homogenized using a high-pressure homogenizer (Emulsiflex C3, Avesin, 950 bar, 4 passes).

### Example 5

### Simulated Digestion of the Powders and Evaluation of Astaxanthin In-Vitro Bioaccessibility

Simulated gastric and intestinal digestion was based on the protocol described by Minekus et al. Food Funct., 2014, 5, 1113-1124. To evaluate AX bioaccessibility, the digested samples underwent ultra-centrifugation (15,000 rpm, 30 min., 20° C.), the upper liquid was collected, and its AX content was determined by extraction and RP-HPLC (reversed-phase HPLC). Extraction by phase separation was achieved by adding dichloromethane and methanol (1:3 v/v), followed by 20 s of vortexing. 2 ml of dichloromethane were then added to each test tube and vortexed for 1 min. The samples were then centrifuged for 10 min at 1,500 G at 4° C. The bottom layer of dichloromethane containing AX was removed and the steps of addition of 2 ml of dichloromethane were repeated twice more. The dichloromethane was evaporated from the extract under a flow of nitrogen. Subsequently, the extract was reconstituted in a known volume of ethanol. Exemplary results of these experiments are represented in FIG. 8.

### Example 6

### Scaled-Up Production of Astaxanthin-Olive Oil-Potato Protein Emulsion

After the identification of the most bio-accessible formulation, changes were made in order to produce the capsules for the clinical trial. AX-Olive oil-PP emulsion was made as described above with slight changes. First, the 4% oil formulation was made using semi-industrial equipment (Nano DeBEE, 1900 bar, 2 passes). In order to increase AX percent in the final powder, the proportions in the formulation were changed to 1:2:3 w/w Oleoresin:Olive oil:PP in the final solution and Tapioca Maltodextrin was added ( 1/20 w/w of the protein amount). Particle size distribution and bioaccessibility was evaluated as described above.

### Example 7

### Clinical Trial for Examining In-Vivo Bioavailability of AX

The in-vivo bioavailability of the most bio-accessible formulation was evaluated and compared to AX oleoresin. The study explored the bioavailability of AX in humans by comparing 2 different formulations which include AX: (A) AX oleoresin; (B) the most bio-accessible formulation of Example 5. The 2 formulations were compared in a single dose (15 mg AX) by 13 volunteers per formulation, in a randomized double-blinded cross-over design, in Rambam Health Care Campus. After a night fast, a blood sample was taken from each volunteer (t=0) and a fat-free yogurt was consumed by the volunteers. After 15 min each volunteer consumed 4 AX capsules (formulation A or B) and blood samples were collected after 2, 4, 6, 8, 10, 24, 48 and 72 hr. The AX in the blood samples was analyzed by RP-HPLC after extraction as described above. The results of the study are summarized in FIGS. 11 and 12. Formulation B significantly increased the bioavailability of AX, as compared to formulation A.

### Example 8

### Encapsulation of CUR-PP, CUR-LEC-PP, CUR-Olive Oil-PP

### Stock Solutions Preparation

PP was dissolved in phosphate buffer (pH=7) at 37° C. for 45 min, while stirring (PP conc. 1 mM). After cooling to room temperature, the solution was centrifuged to precipitate insoluble matter (3000 rpm, 5 min). The supernatant was collected and filtered by vacuum filtration (0.45 μm filter). The final protein conc. was determined by spectrophotometer (277 nm). Pure curcumin (CUR) powder (95%) was dissolved in absolute ethanol at 40° C. for 30 min, while stirring, to obtain ethanolic CUR stock solution (7.5 mM). Sunflower LEC was dissolved in 20 ml of absolute ethanol for 2 hr (LEC conc. 27 mM). The solution was centrifuged for 10 min at 5000 rpm. The supernatant containing the ethanol soluble fraction of LEC was collected. The final conc. was determined gravimetrically by evaporating the ethanol from the supernatant and was found to be 12.8 mM.

### CUR-PP Nanoparticles Formation

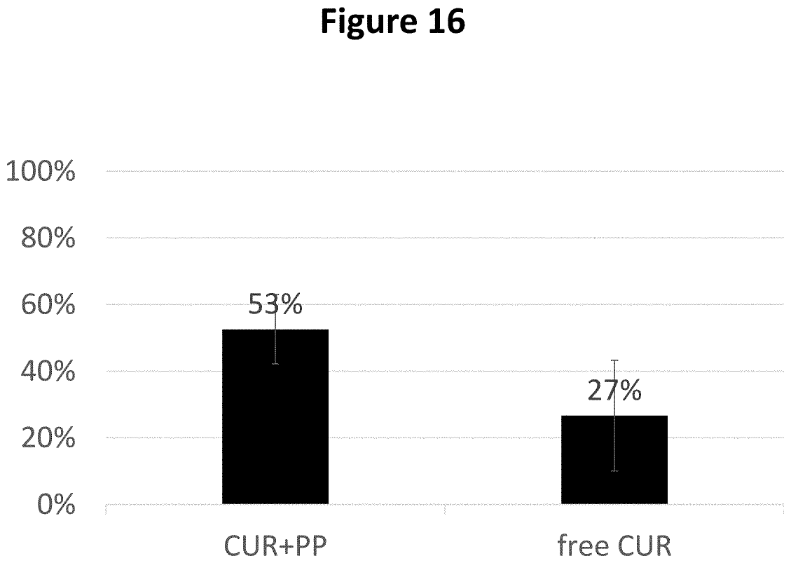

An ethanolic stock solution of curcumin powder was added dropwise into PP solution, during vortex, and left shaken for 1 hr. CUR concentration was 1.5 mM in all samples. Exemplary results, showing enhanced bioaccessibility of curcumin, are depicted by FIG. 16.

### CUR-LEC-PP Nanoparticles Formation

Pure curcumin (CUR) powder (95%) was dissolved in LEC ethanolic solution at 40° C. for 30 min, while stirring (equal concentration of CUR and LEC, 10 mM each). The CUR-LEC ethanolic solution was added dropwise into PP solution, during vortex and left shaken for 1 hr. CUR concentration was 1.5 mM in all samples.

### CUR-Olive Oil-PP Emulsion Preparation

CUR-PP nanoparticles were formed as mentioned above, in 9:1 molar ratio (CUR:PP, 0.06%:0.73% w/v in buffer). After 1 hr. shaking, the NPs solution was mixed with olive oil and pre-homogenized using a desktop homogenizer (30,000 rpm, 5 min) (final conc. of the oil was 0.73% w/v in the buffer). The pre-emulsion obtained was homogenized using a high-pressure homogenizer (Emulsiflex C3, Avesin, 950 bar, 4 passes).

The abovementioned particles (CUR-LEC-PP, CUR-Olive oil-PP) undergo biological studies, so as to evaluate the potential bioaccessibility and/or bioavailability of these molecules versus non-encapsulated curcumin. It is postulated, that such particles will exhibit an increased bioaccessibility and/or bioavailability, as well-known from studies performed on curcumin encapsulated by PP, and as represented by FIG. 17 showing an increased protection of the encapsulated curcumin (Curcumin-PP-Lecithin) as evaluated in the simulated digestion model (described hereinabove in Example 5).

### Free CUR Sample Preparation (Control)

An ethanolic stock solution of curcumin powder was added dropwise into phosphate buffer, during vortex, and left shaken for 1 hr. CUR concentration was 1.5 mM in all samples.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. (canceled)

2. A particle comprising a plant oil encapsulated in an amphiphilic shell, wherein said amphiphilic shell comprises an amphiphilic plant protein and a hydrophobic compound, wherein the w/w ratio of said hydrophobic compound to said plant protein ranges from 0.01:1 to 1:1, and wherein the w/w ratio of said plant protein to said plant oil ranges from 0.1:1 to 10:1.

3. The particle of claim 2, wherein said hydrophobic compound has a low solubility in said plant oil and is optionally selected from the group consisting of: a phenolic compound, a tannin, a stilbene, a curcuminoid, a coumarin, a lignan, a quinone, or any combination thereof.

4. The particle of claim 1, wherein said shell is a single layer shell.

5. The particle of claim 2, comprising: a carotenoid, a flavonoid, a phytosterol, an antioxidant, a phytoestrogen, a polyphenol, a cannabinoid, a hydrophobic drug, a hydrophobic nutraceutical, and anthocyanin or any combination thereof.

6. (canceled)

7. The particle of claim 2, wherein (a) said plant oil is selected from the group consisting of: an olive oil, a triglyceride oil, a terpenoid oil, a sunflower oil, a nut oil, a peanut oil, a soy oil, a rapeseed oil, a sesame oil, a palm oil, a cocoa butter, a rice oil, a wheat germ oil, a citrus oil, limonene or any combination thereof, (b) said amphiphilic plant protein is selected from the group consisting of: a potato protein (PP), a sweet potato protein, a soy protein, a rice protein, a wheat protein, a legume protein, a cereal protein, an algal protein, a hydrolyzed soy protein, a hydrolyzed rice protein, a hydrolyzed wheat protein, a hydrolyzed cereal protein, a hydrolyzed algal protein and a hydrolyzed legume protein or any combination thereof, or (a) and (b).

8. The particle of claim 2, wherein said amphiphilic plant protein is selected from the group consisting of: a potato protein (PP), a sweet potato protein, a soy protein, a rice protein, a wheat protein, a legume protein, a cereal protein, an algal protein, a hydrolyzed soy protein, a hydrolyzed rice protein, a hydrolyzed wheat protein, a hydrolyzed cereal protein, a hydrolyzed algal protein and a hydrolyzed legume protein or any combination thereof.

9. (canceled)

10. (canceled)

11. The particle of claim 2, wherein said hydrophobic compound comprises said curcuminoid, and said plant oil comprises said olive oil.

12. The particle of claim 11, wherein a w/w ratio of said curcuminoid to said olive oil is between 0.1:1 and 1:1; and wherein a w/w ratio of said PP to said olive oil is between 3:1 and 1:3.

13. (canceled)

14. A particle comprising a hydrophobic core and an amphiphilic shell, wherein said hydrophobic core comprises a hydrophobic compound, wherein said amphiphilic shell comprises a first layer and a second layer, wherein said first layer comprises a surfactant and said second layer comprises an amphiphilic plant protein, wherein the w/w ratio of said amphiphilic plant protein to said surfactant ranges from 1:1 to 500:1, and wherein the w/w ratio of said hydrophobic compound to said surfactant ranges from 0.01:1 to 1:10 of said particle.

15. The particle of claim 14, wherein said hydrophobic compound (i) is selected from the group consisting of: carotenoid, a flavonoid, a phytosterol, an antioxidant, a phytoestrogen, a polyphenol, and anthocyanin or any combination thereof; or (ii) has a low solubility in said plant oil and is optionally selected from the group consisting of: a phenolic acid, a tannin, a stilbene, a curcuminoid, a coumarin, a lignan, a quinone, or any combination thereof.

16. The particle of claim 14, wherein said plant oil is selected from the group consisting of: an olive oil, a triglyceride oil, a terpenoid oil, a citrus oil, a sunflower oil, a peanut oil, a soy oil, a rapeseed oil, a soybean oil, a palm oil, a cocoa butter, a rice bran oil, and limonene or any combination thereof.

17. The particle of claim 14, wherein said plant oil comprises said olive oil; said hydrophobic compound comprises curcumin, AX or both; and said surfactant comprises lecithin.

18. The particle of claim 14, wherein said amphiphilic plant protein is selected from the group consisting of: a potato protein (PP), a sweet potato protein, a soy protein, a rice protein, a wheat protein, a legume protein, a cereal protein, an algal protein, a hydrolyzed soy protein, a hydrolyzed rice protein, a hydrolyzed wheat protein, a hydrolyzed cereal protein, a hydrolyzed algal protein and a hydrolyzed legume protein or any combination thereof.

19. (canceled)

20. (canceled)

21. A composition comprising the particle of claim 14, and an aqueous solution, wherein said hydrophobic compound is at a concentration of 1 to 10000 ppm in said composition.

22. (canceled)

23. A method for solubilizing a hydrophobic compound in an aqueous formulation, comprising: (i) mixing said hydrophobic compound and a plant oil at a w/w ratio ranging from 0.01:1 to 1:1 at 30-70° C., to obtain an oil phase, (ii) providing an aqueous solution comprising an aqueous phosphate buffer and an amphiphilic plant protein, (iii) adding said aqueous solution to said oil phase to obtain a final solution, (iv) homogenizing said final solution, thereby solubilizing said hydrophobic compound in said aqueous formulation.

24. (canceled)

25. (canceled)

26. The method of claim 23, further comprising freeze-drying said aqueous formulation to obtain a powder, thereby obtaining a powderous composition comprising said hydrophobic compound.

27. The method of claim 26, further comprising mixing said powderous composition with an aqueous solution, thereby obtaining a reconstituted aqueous formulation comprising said hydrophobic compound.

28. (canceled)

29. (canceled)

30. (canceled)

31. (canceled)
